(19) United States
(12) Patent Application Publication
(10) Pub. No.: US 2021/0178142 A1
(43) Pub. Date: Jun. 17, 2021

(54) ADJUSTABLE CANNULATION ASSEMBLY AND METHODS THEREOF

(71) Applicant: LSI Solutions, Inc., Victor, NY (US)

(72) Inventors: Jude S. Sauer, Pittsford, NY (US); Jeremy A. Schiele, Fairport, NY (US)

(21) Appl. No.: 17/121,025

(22) Filed: Dec. 14, 2020

Related U.S. Application Data
(60) Provisional application No. 62/948,029, filed on Dec. 13, 2019.
(60) Provisional application No. 63/062,646, filed on Aug. 7, 2020.
(60) Provisional application No. 63/077,388, filed on Sep. 11, 2020.

(51) Int. Cl. A61M 39/10; A61M 1/36; A61M 1/16; A61M 39/20; A61M 25/09

(52) U.S. Cl. CPC A61M 39/105; A61M 1/3659; A61M 2207/00; A61M 39/20; A61M 25/09041; A61M 1/1698

(57) ABSTRACT

A cannulation coupler has a primary branch comprising a retaining feature. The cannulation coupler also has a side inflow branch in communication with the primary branch. The cannulation coupler further has an outflow branch in communication with the primary branch and in communication with the side inflow branch. An adjustable cannulation assembly is also disclosed, having a cannulation coupler, an outer cannula, and an inner cannula. The cannulation coupler has 1) a primary branch comprising a retaining feature, 2) a side inflow branch in communication with the primary branch, and 3) an outflow branch in communication with the primary branch and in communication with the side inflow branch. The outer cannula is coupled to the primary branch. The inner cannula is coupled to the side inflow branch, wherein: 1) the inner cannula also passes through the primary branch; and 2) the inner cannula is coaxially slidable within the outer cannula.

### RELATED APPLICATIONS

This application claims priority to U.S. Provisional Patent Application No. 62/948,029 filed Dec. 13, 2019 and entitled, “ADJUSTABLE CANNULATION ASSEMBLY AND METHODS THEREOF”. This application also claims priority to U.S. Provisional Patent Application No. 63/062,646 filed Aug. 7, 2020 and entitled, “ADJUSTABLE CANNULATION ASSEMBLY AND METHODS THEREOF”. This application further claims priority to U.S. Provisional Patent Application No. 63/077,388 filed Sep. 11, 2020 and entitled, “ADJUSTABLE CANNULATION ASSEMBLY AND METHODS THEREOF”. Each of the 62/948,029, 63/062,646, and 63/077,388 applications are hereby incorporated by reference in their entirety.

### FIELD

The claimed invention relates to cannulation assembly for minimally invasive cardiac interventions, and more specifically to an adjustable cannulation assembly for minimally invasive cardiac interventions.

### BACKGROUND

Extracorporeal membrane oxygenation (ECMO) is utilized as a temporary form of mechanical circulatory support and simultaneous gas exchange for patients with cardiogenic shock or refractory heart failure, for example. In addition to providing a patient with circulatory support, ECMO may allow time for other treatments, promote recovery, or act as a bridge to alternate, more durable mechanical solutions to address acute or chronic cardiopulmonary failure. Typical ECMO circuits include a venous or return or outflow cannula, a pump, an oxygenator, and an arterial or inflow cannula.

A number of approaches can be utilized with an ECMO system, including via the apex of the heart for left-sided support (VA-ECMO) and via the right or left internal jugular vein for right-sided and/or respiratory support (VV-ECMO). Various forms of peripheral ECMO may involve femoro-femoral access, internal jugular access, or internal jugular vein access with return to a graft placed on the subclavian artery. These forms of ECMO, while effective, may present issues with mobility, issues with access site infection, in particular with the femoro-femoral access, as well as issues with rendering the patient non-ambulatory during the ECMO intervention and related procedures. These issues may adversely impact the healing process.

Transapical cannula placement into the left ventricle (VA-ECMO) can be used for patients requiring ECMO. Transapical cannula placement into the left ventricle in the setting of VA-ECMO for refractory heart failure normally requires sternotomy or thoracotomy in a highly vulnerable patient, which carries a significant risk of bleeding. VV-ECMO with access via the right or left internal jugular vein is also used, however, these approaches do not come without significant morbidity/mortality and advances must be made to maximize clinical benefits and minimize risk. Minimally invasive approaches are under development but are typically fixed in size and designed with healthy patients in mind or for patients with more predictable anatomical features in regard to the location, size, and shape of the heart. Therefore, there is a need for cannulation systems applicable to transapical and/or internal jugular vein cannula placement for use with ECMO that are adjustable based on patient size and anatomical variations. Such a customizable, patient-centered system would further enable cannula placement while allowing patients to ambulate on ECMO and therefore improve morale, hasten recovery, reduce morbidity, and optimize patient outcomes.

### BRIEF DESCRIPTION OF THE DRAWINGS

FIG. 1 is a top-left-front perspective view of a cannulation coupler.

FIGS. 2A, 2B, 2C, 2D, 2E, and 2F are front, left side, right side, rear, top, and bottom elevational views, respectively, of the cannulation coupler of FIG. 1.

FIG. 3 is an exploded view illustrating assembly of the of the cannula coupler of FIG. 1.

FIG. 4 is a cross-sectional view of a cannulation assembly utilizing the cannula coupler of FIG. 1.

FIG. 5 is a partial cross-sectional view of a heart with a cannulation assembly inserted.

FIG. 6 is a schematic view of a surgical setting employing the cannula coupler assembly of FIG. 5.

FIG. 7 is a top-left-front perspective view of another embodiment of a cannulation coupler suitable for use in a single cannula dual lumen adjustable cannulation assembly for minimally invasive ambulatory ECMO.

FIGS. 8A, 8B, 8C, 8D, 8E, and 8F are front, left side, right side, rear, top, and bottom elevational views, respectively, of the cannulation coupler of FIG. 7.

FIGS. 9A-9C are a series of exploded views showing assembly steps of an adjustable cannulation assembly including the cannulation coupler of FIG. 7.

FIG. 10 is an exploded view illustrating assembly of the of the cannula coupler of FIG. 7.

FIG. 11 is a schematic view of a surgical setting employing the cannula coupler assembly illustrated in FIGS. 9A-9C.

FIG. 12 is a perspective view of another embodiment of a cannulation coupler suitable for use in a single cannula dual lumen adjustable cannulation assembly for minimally invasive ambulatory ECMO.

FIG. 13 is a top-left-front perspective view of an adjustable cannulation assembly.

FIG. 14 is an exploded perspective view of the adjustable cannulation assembly of FIG. 13.

FIG. 15 is a top-left-front perspective view of a pulmonary artery guidewire director for use accompanying the adjustable cannulation assembly of FIG. 13.

FIG. 16 is a top-left-front perspective view of an inner cannula for use accompanying the adjustable cannulation assembly of FIG. 13.

FIG. 17 is a top-left-front perspective view of an inner cannula obturator for use accompanying the adjustable cannulation assembly of FIG. 13.

FIGS. 18A and 18B are top-left-front and bottom-right-rear perspective views, respectively, of the pulmonary artery guidewire introducer of the adjustable cannulation assembly of FIG. 13.

FIGS. 19A-19H and 19J-19N are schematic illustrations of a surgical method for use of the adjustable cannulation assembly of FIG. 13 with the additional components of FIG. 15, FIG. 16, and FIG. 17. It should be noted that FIG. 19I was not used so as not to be confused with the number 191.

FIG. 20 is a side view of the adjustable cannulation assembly of FIG. 13 illustrating several locations along a path followed by a directed pulmonary artery guidewire through the adjustable cannulation assembly.

FIGS. 21A-21F are a series of several cross-sectional views of the adjustable cannulation assembly indicated in FIG. 20.

FIGS. 22A and 22B are side views of one embodiment of an adjustable cannulation assembly illustrating adjustment of the second lumen relative to the first lumen.

It will be appreciated that for purposes of clarity and where deemed appropriate, reference numerals have been repeated in the figures to indicate corresponding features, and that the various elements in the drawings have not necessarily been drawn to scale in order to better show the features.

### DETAILED DESCRIPTION

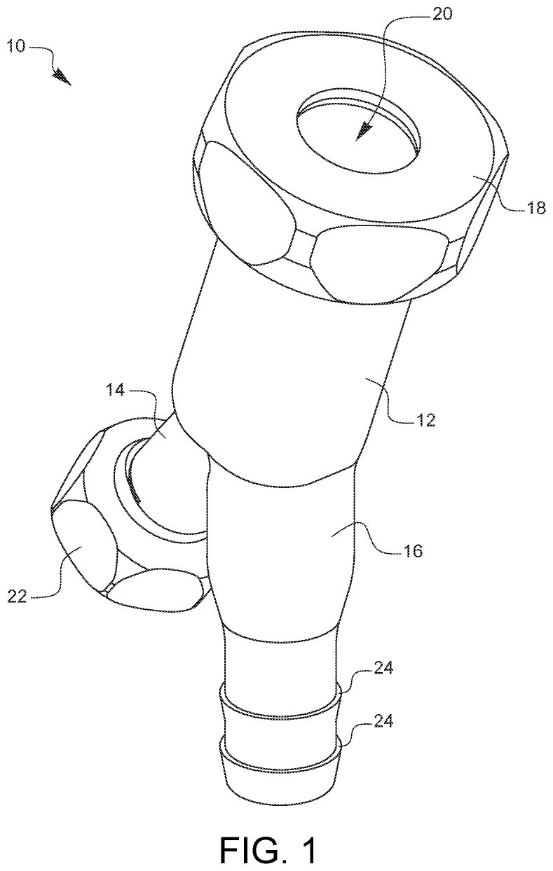

FIG. 1 is a top-left-front perspective view of a cannulation coupler suitable for use in a single cannula dual lumen adjustable cannulation assembly for minimally invasive ambulatory VA-ECMO. The cannulation coupler 10 has a primary branch 12 with a primary opening 20 in communication with a side inflow branch 14 and an outflow branch 16. The primary branch 12 has a primary compression cap 18 configured to hold and secure a dual lumen cannula in the cannulation coupler 10 during use. The cannulation coupler 10 also has an inflow compression cap 22 on the inflow branch 14, which is configured to hold and secure an inflow or inner cannula. The cannulation coupler 10 may be fabricated from a number of materials suitable for surgical use including surgical steel, plastic, or other suitable materials known in the art for transferring blood or similar fluids without interacting with the fluids unfavorably. It should be noted that the diameter of the inflow branch 14 is smaller than the primary branch 12. Other embodiments of cannulation couplers may have a larger inflow branch than the primary branch. Still other embodiments of cannulation couplers may have inflow and primary branches having substantially similar diameters.

In an effort to clarify terminology, various descriptions have been used to characterize or describe the flow or direction of blood from the perspective of the cannulation coupler. The inflow cannula and the inflow branch of the cannulation coupler carries arterial or oxygenated blood from the ECMO system. The outflow cannula and the outflow branch, also referred to as a drainage cannula or drainage branch, of the cannulation coupler carries venous or deoxygenated blood back to the ECMO system for the purpose of oxygenating the blood flow. A primary branch merges an inflow and outflow branch or cannula into a dual lumen coaxial configuration. Other conventions in terminology, particular in reference to reversing the order of terminology relating to flow direction may exist within the medical community, but for the purposes of this description, the terminology will be used as noted above.

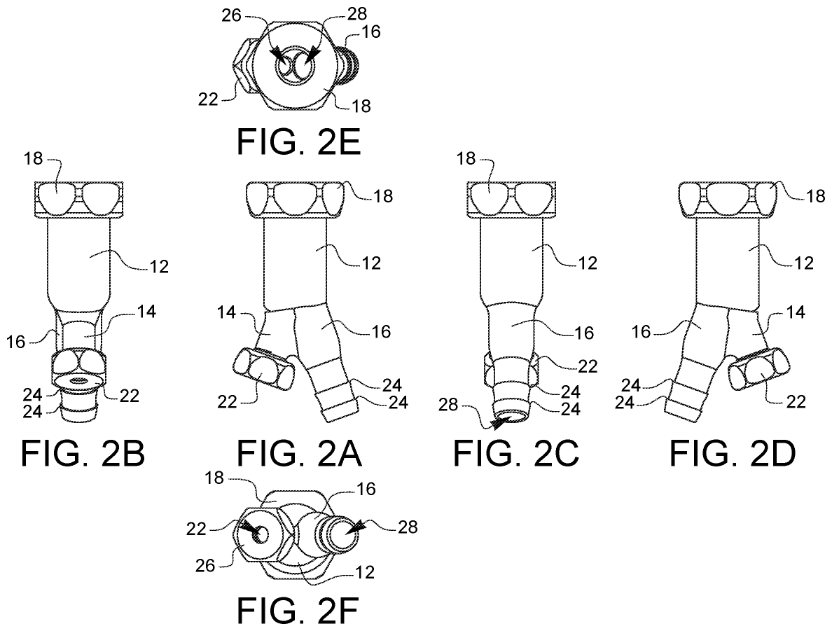

FIGS. 2A, 2B, 2C, 2D, 2E, and 2F are front, left side, right side, rear, top, and bottom elevational views, respectively, of the cannulation coupler of FIG. 1. FIGS. 2E and 2F, in particular, illustrate the respective locations and orientations of the inflow opening 26 and the outflow opening 28 with the inflow compression cap 22 assembled and attached.

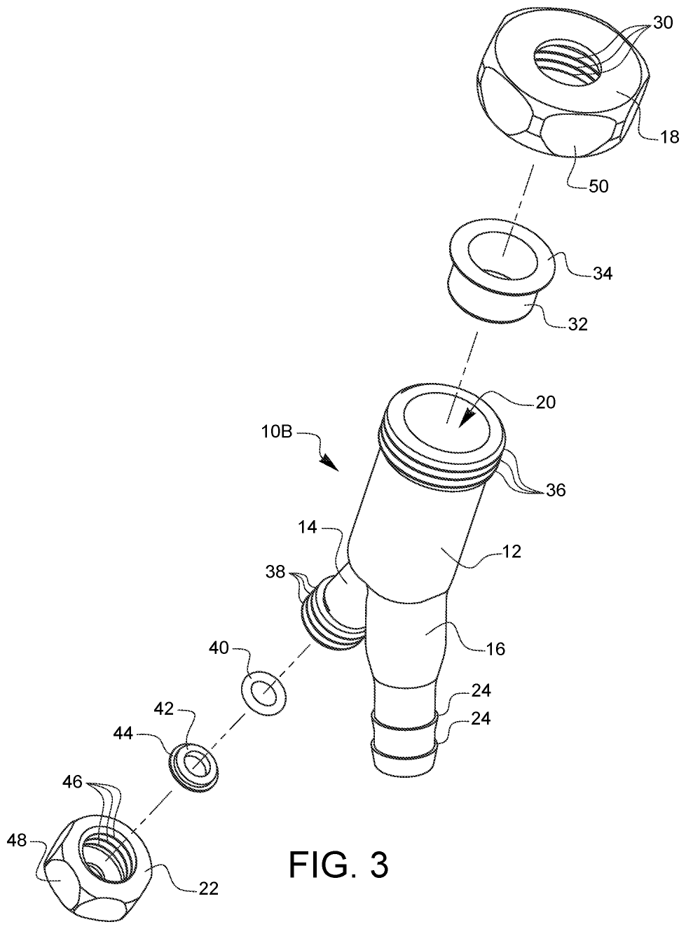

FIG. 3 is an exploded view illustrating assembly of the of the cannula coupler of FIG. 1. The body 10B of the cannulation coupler 10 defines helical threads 36 on an outside circumference of the primary branch 12, near the primary opening 20. A washer 32 having a step 34 to provide a depth limiter is inserted into the primary opening 20 of the primary branch 12. The primary compression cap 18 has helical inner threads 30 that correspond to the threads 36 on the body 10B of the cannulation coupler 10 and flats 50 on the outer circumference of the primary compression cap 18. The flats 50 provide a configuration for tightening of the primary compression cap 18 by mechanical tools, although the primary compression cap 18 may also be tightened by hand. The primary compression cap 18 is not fully tightened until a cannula or lumen is also inserted into the primary opening 20 once the washer 32 and primary compression cap 18 are attached to the primary branch 12. Once fully tightened, the primary compression cap 18 and primary washer 32 provide a leakproof and hermetically sealed connection for holding an outer dual lumen cannula providing a flow pathway through the connected outer cannula. On the end of the outflow branch 16, the outflow branch 16 defines several barbs 24. These barbs 24 are configured to temporarily yet reliably hold an outflow tube or lumen used in an ECMO surgical assembly and apparatus. On the inflow branch 14 of the body 10B of the cannulation coupler 10 are a set of helical threads 38. The inner diameter of the inflow branch 14 is configured such that a sealing element such as an o-ring 40 can be inserted into the inflow branch 14 without the o-ring 40 falling into the opening of the inflow branch 14. This may be a step or a ledge on the inner diameter of the inflow branch 14. This feature is not shown here, but should be known to those skilled in the art. Once the o-ring 40 is in place inside the inflow opening 26 of the inflow branch 14, an inflow washer 42 is also placed in the inflow branch 14. The inflow washer 42 has a step 44 to limit the depth of its insertion into the inflow opening 26 of the inflow branch 14. The inflow compression cap 22 also defines inner threads 46 on an inner circumference and several flats 48.

The flats 48 on the inflow compression cap 22 provide a configuration for tightening of the inflow compression cap 22 by mechanical tools, although the inflow compression cap 22 may also be tightened by hand. The inflow compression cap 22 is not fully tightened until a cannula or lumen is also inserted into the inflow opening 26 once the o-ring 40, washer 42 and the inflow compression cap 22 are attached to the inflow branch 14. Once fully tightened, the inflow compression cap 22, o-ring 40, and washer 42 provide a leakproof and hermetically sealed connection for holding an inner cannula providing a flow pathway into the cannulation coupler 10.

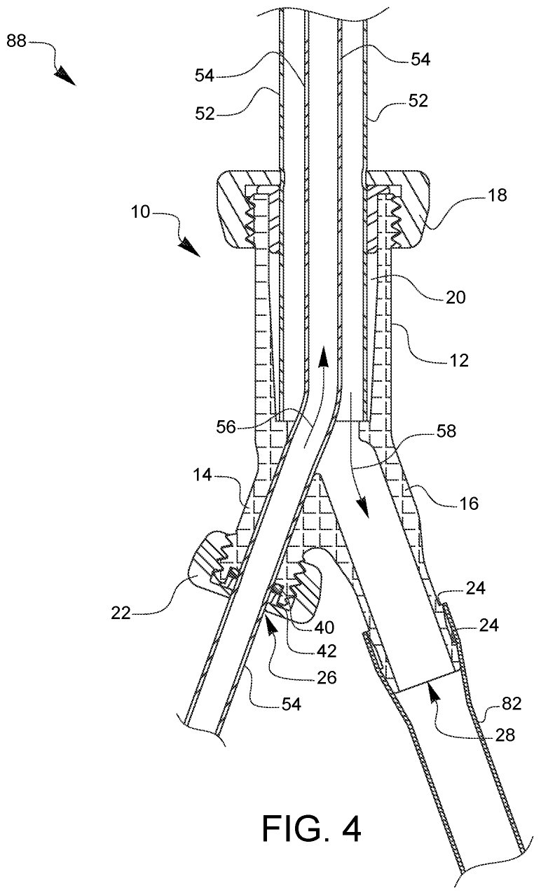

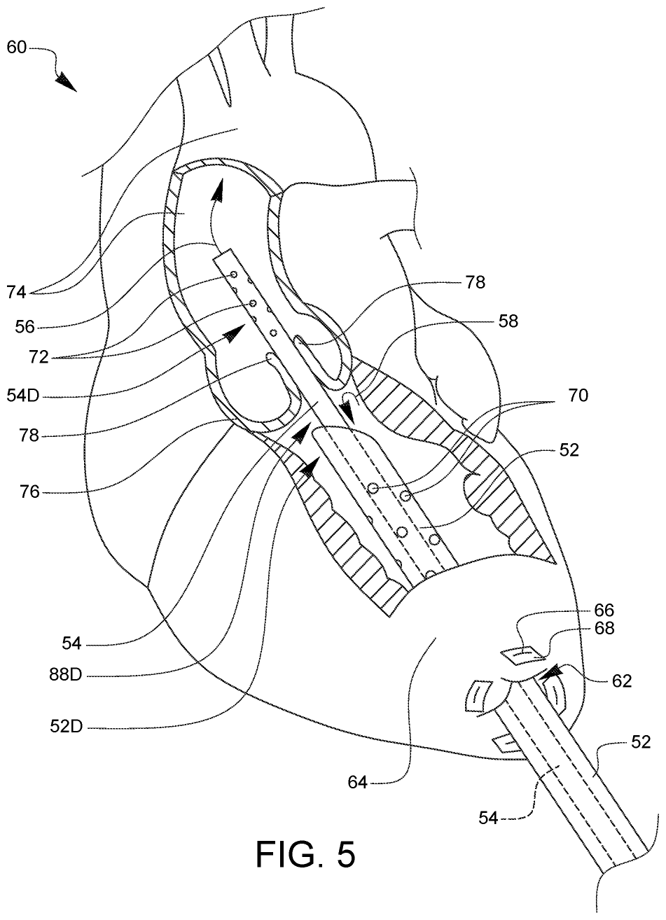

FIG. 4 is a cross-sectional view of a cannulation assembly utilizing the cannula coupler of FIG. 1. An outer lumen or outer cannula 52 is shown inserted into the primary opening 20 and primary branch 12 end of the cannulation coupler 10 with the washer 32 in place and the primary compression cap 18 tightened and sealed. An inner lumen or inner cannula 54 is also shown inserted into the inflow opening 26 of the cannulation coupler 10 with the o-ring 40 in place and the inflow compression cap 22 tightened and sealed. The inner cannula 54 is further inserted into the outer cannula 52 through the primary branch 12 resulting in a dual lumen configuration. This could also be characterized as a coaxial dual lumen configuration or a cannula-in-cannula or lumen-in-lumen configuration. FIG. 4 also illustrates the inflow direction 56 of the cannulation assembly 88, which demonstrates the pathway and flow direction of the arterial blood into the aorta carried by the inner cannula 54. Also illustrated is the pathway and outflow direction 58 of the venous or return flow carried by the outer cannula 52 in an ECMO system. These pathways and their overall system configuration will be discussed further in regard to FIG. 5.

FIG. 5 is a partial cross-sectional view of a heart with a cannulation assembly inserted into a heart. A patient's heart 60 is shown with the distal end 88D of the cannulation assembly 88 shown in its intended placement within the heart 60. The distal end 88D of the cannulation assembly 88 is inserted into an apical opening 62 in the left ventricle 64 of the heart 60. The cannulation assembly 88 is secured to the heart 60 with several sutures 66 and supporting pledgets 68 surrounding the apical opening 62. As inserted, the coaxial dual lumen cannula is configured such that the distal end 52D of the outer cannula 52 is located in the left ventricle 64. This allows the deoxygenated blood to flow in outflow direction 58 into the distal end 52D of the outer cannula 52 back to the ECMO system for oxygenation. The distal end 52D of the outer cannula 52 also defines several perforations 70 that further enable adequate blood flow should the distal end of the outer cannula 52 be pressed against the inner wall of the heart. The distal end 54D of the inner cannula 54 is inserted further into the heart 60, such that the distal end 54D passes through an aortic valve 76 to deliver oxygenated blood from the inner cannula 54 into an aorta 74. When inserted into the aorta 74 the inner cannula 54 is sealed relative to the left ventricle 64 by coaptation of aortic valve leaflets 78 of the aortic valve 76. This effectively isolates the inflow of oxygenated blood from the inner cannula 54 into the aorta 74 from the outflow of deoxygenated blood from the apical opening 62 out from the left ventricle 64. The inner cannula 54 further defines several perforations 72 at the distal end 54D that further enable adequate blood flow should the distal end of the inner cannula 54 be otherwise obstructed. Since the coaxial dual lumen set up is further configured such that the inner cannula 54 can be slidable coaxially from a proximal direction to a distal direction, and vice versa within the outer cannula 52, the relative position of the distal end 54D of the inner cannula 54 can be adjustable in relation to the position of the 52D distal end of the outer cannula 52. This enables an adjustability not available to conventional cannulation assemblies that have fixed positions and are not coaxially oriented relative to an inner and outer lumen single cannula. This adjustability is important to accommodate variance in anatomical sizing and features that may be present across different patients. It should also be noted that while being carried in a single, dual lumen coaxial cannula, the inflow and the outflow pathways do not blend or cross contaminate. While a transapical approach is shown, other introductory methods including VA-ECMO, VV-ECMO, and others may be utilized with such an adjustable cannula assembly as described herein.

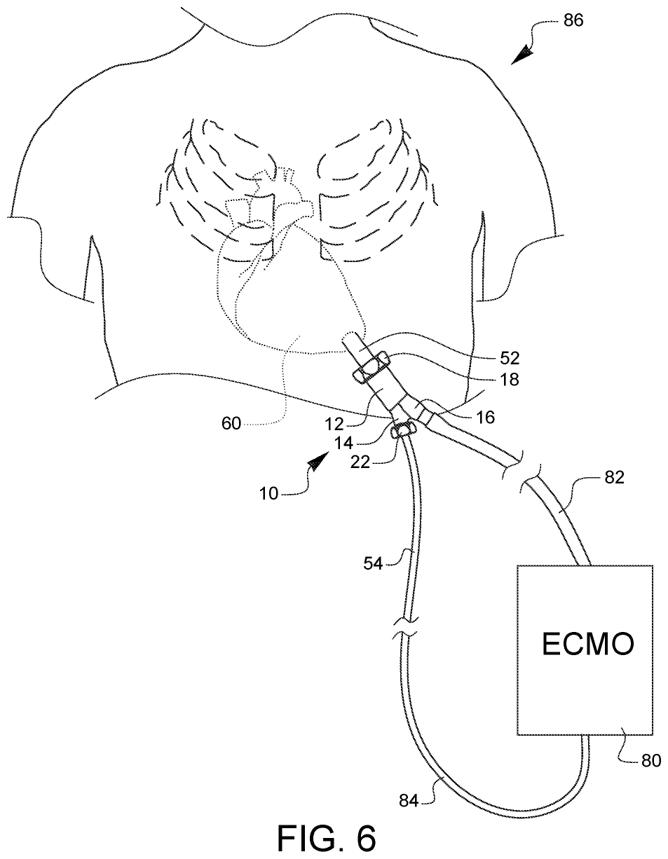

FIG. 6 is a schematic view of a surgical setting employing the cannula coupler assembly of FIG. 5. FIG. 6 illustrates the relative positioning of a patient 86 and the cannulation assembly 88 as inserted. An inflow lumen 84 connected to the inner cannula 54 is connected to the inflow branch 14 of the cannulation coupler 10 and carries oxygenated blood from the ECMO apparatus 80 to the patient 86. The outer portion of the outer cannula 52 brings deoxygenated blood out of the patient and into the ECMO apparatus 80 for oxygenation via an outflow lumen 82. The cannulation coupler 10 may be secured to the patient 86 externally in order to facilitate ambulatory movement of the patient 86 while undergoing treatment.

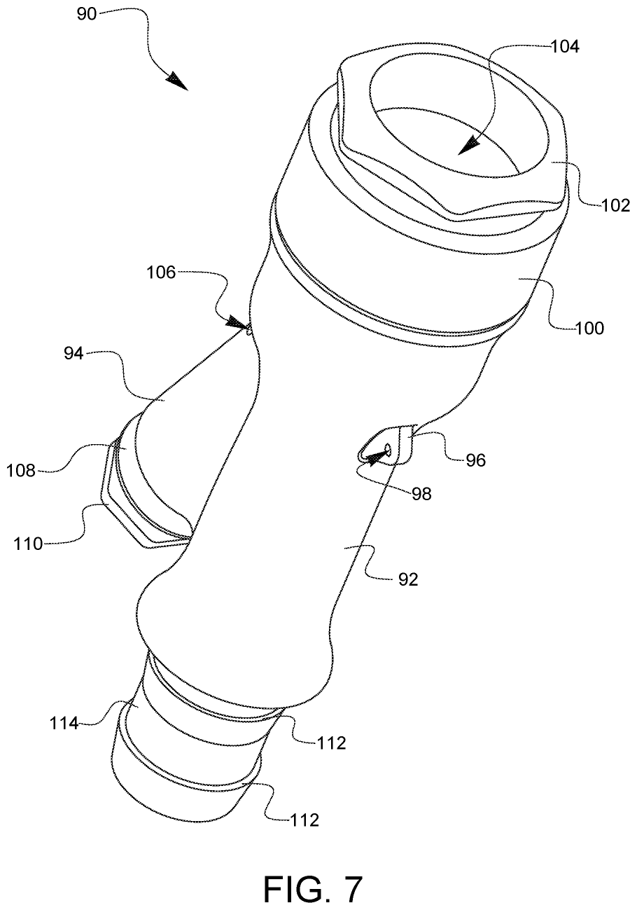

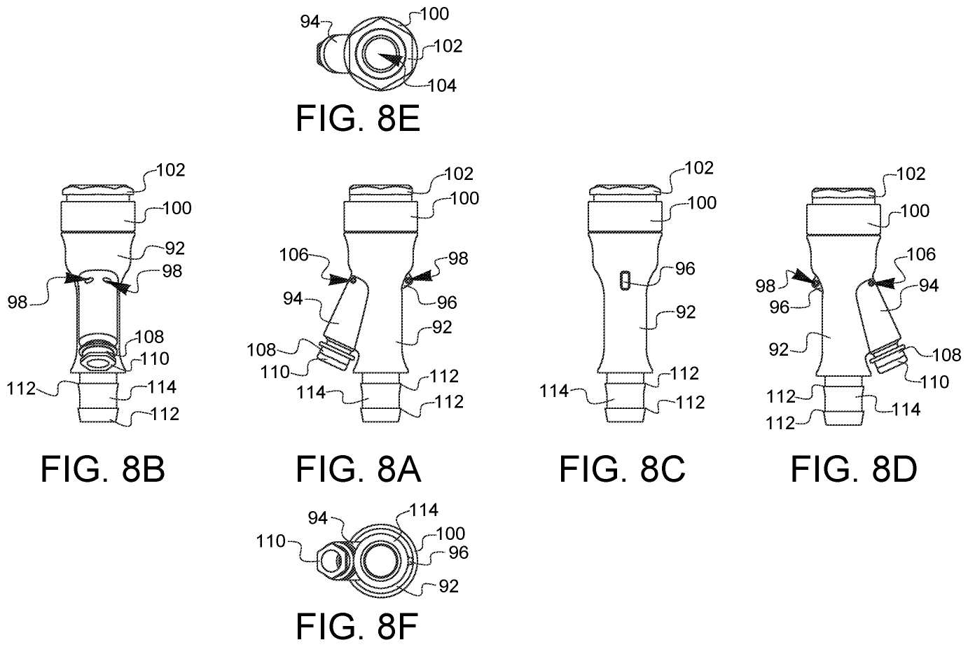

FIG. 7 is a top-left-front perspective view of another embodiment of a cannulation coupler suitable for use in a single cannula dual lumen adjustable cannulation assembly for minimally invasive ambulatory ECMO. The coaxial coupler or cannulation coupler 90 has a base port section or primary branch 92 with a base port opening 104 in communication with an inflow side port 94 and an external connection end or outflow branch 114. The primary branch 92 has a base port cap 100 configured to hold and secure a dual lumen cannula in the cannulation coupler 90 during use. The base port cap 100 has a head 102 which is configured to assist the user in fastening the base port cap 100 onto the threaded end of the primary branch 92 with the use of an adjustable wrench or similar tool. While the head 102 illustrated is a hex-type nut shape, other fastening head shapes or configurations known to those skilled in the art may also be utilized. The cannulation coupler 90 also has side cap 108 on the inflow side port 94, which is configured to hold and secure an inflow or inner cannula to the inflow side port 94. The side cap 108 has a head 110 which is configured to assist the user in fastening the side cap 108 onto the threaded end of the inflow side port 94 with the use of an adjustable wrench or similar tool. While the head 110 illustrated is a hex-type nut shape, other fastening head shapes or configurations known to those skilled in the art may also be utilized. The outflow branch 114 has several concentric barbs 112 defined by the outflow branch 114. These barbs 112 are configured to secure a tube or other cannula which may be placed onto the outflow branch 114 of the cannulation coupler 90. As this embodiment illustrates barbs 112 on the outflow branch 114, other securing means known in the art may be used in the assembly. The cannulation coupler 90 may be fabricated from a number of materials suitable for surgical use including surgical steel, plastic, or other suitable materials known in the art for transferring blood or similar fluids without interacting with the fluids unfavorably. It should be noted that the diameter of the inflow side port 94 is smaller than the primary branch 92. Other embodiments of cannulation couplers may have a larger inflow side port than the primary branch. Still other embodiments of cannulation couplers may have inflow side port and primary branches having substantially similar diameters or sizes. A protrusion 96 defined by the primary branch 92 further defines a retaining feature, an attachment eyelet 98 configured to secure the cannulation coupler 90 to a patient once the cannulation coupler 90 and its accompanying assembly is installed and completed. A second inflow side port retaining feature or attachment eyelet 106 is also defined by the inflow side port 94. FIGS. 8A, 8B, 8C, 8D, 8E, and 8F are front, left side, right side, rear, top, and bottom elevational views, respectively, of the cannulation coupler of FIG. 7.

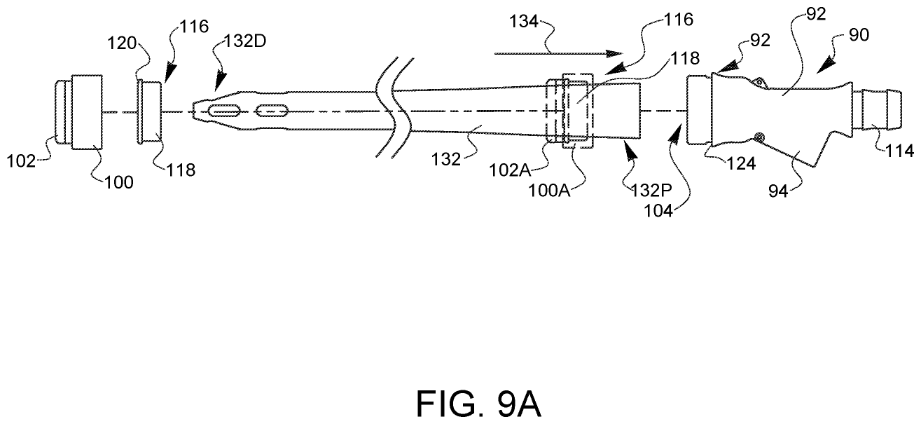

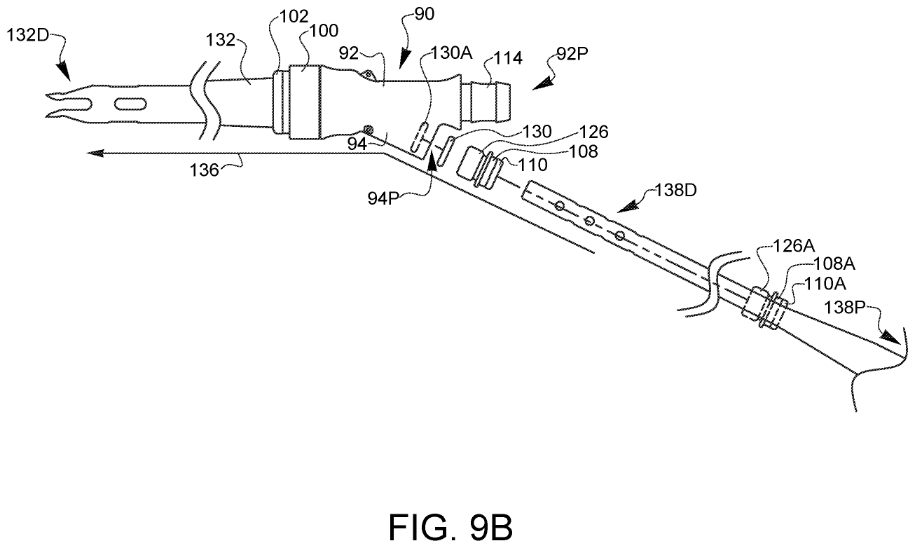

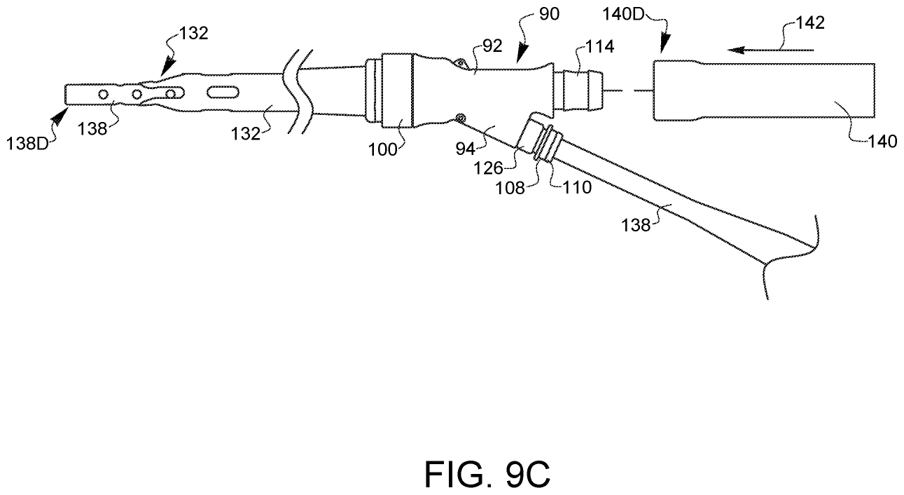

FIGS. 9A-9C are a series of exploded views showing assembly steps of an adjustable cannulation assembly including the cannulation coupler of FIG. 7. These assembly steps are shown outside the context of patient or instrumentation use for purposes of clarity. As shown in FIG. 9A, a first lumen 132 has been modified by cutting or otherwise removing a portion of a distal end 132D proximal end 132P of the first lumen 132 until approximately 2 mm from the reinforcement end is remaining on the distal end 132D proximal end 132P of the first lumen 132. While not shown in this view, a portion of a distal end 132D of the first lumen 132 may also be removed in order to facilitate subsequent steps of the assembly. This sectioning of a portion of the distal end 132D of the first lumen 132 can be accomplished by beginning to section at or near a port hole in a distal end 132D of a first lumen 132, then cutting towards the distal end along a center line of the lumen. The distal end 132D can be further flared and any excess material or sharp edges removed. Next, the bushing 116 is placed over the distal end 132D of the first lumen 132 and moved towards the distal end 132D proximal end 132P of the first lumen 132 in direction 134. The base port cap 100 is then placed over the distal end 132D of the first lumen 132 and moved towards the distal end 132D proximal end 132P of the first lumen 132 in direction 134. The distal end 132D proximal end 132P of the first lumen 132 and the insert 118 on the bushing 116 are then placed into the base port opening 104 of the cannulation coupler 90 such that the flange 120 of the bushing 116 is contacting the primary branch attachment cylinder 122 and the distal end 132D proximal end 132P of the first lumen 132 is fully seated and sealed in the base port opening 104 of the cannulation coupler 90. The base port cap 100 is then tightened by hand or with an additional tool to fully fasten the base port cap 100 onto the cannulation coupler 90 and seal the first lumen 132 into the cannulation coupler 90. The result of the preceding assembly steps is shown in FIG. 9B. Next, an o-ring 130 is inserted and seated into a proximal end 94P of inflow side port 94 of the cannulation coupler 90. Side cap 108 is placed over a distal end 138D of a second lumen 138 and slid towards a proximal end 138P of the second lumen 138. The distal end 138D of the second lumen 138 is then inserted into the proximal end 94P of the inflow side port 94, through the primary branch 92 and into the distal end 132D proximal end 132P of the first lumen 132 towards the distal end 132D of the first lumen 132 in direction 136. Now the portion of the first lumen 132 protruding from the base port cap 100 of the cannulation coupler 90 has the second lumen 138 coaxially inserted throughout its length and the distal end 138D of the second lumen 138 is protruding from the distal end 132D of the first lumen 132, as illustrated in FIG. 9C. FIG. 9C shows the result of the preceding assembly steps. A last assembly step shows the attachment of an external lumen 140 by placing a distal end 140D of the external lumen 140 in direction 142 over the outflow branch 114. While this method and order of component assembly has been described in regard to FIGS. 9A-9C, other means and orders of operation may be used in order to achieve the same structure and function of an adjustable cannulation assembly. The removing of a section from a proximal end of a first cannula, removing of a section from a distal tip of the first cannula, affixing the proximal end of the first cannula onto a main port of a cannulation coupler, inserting a distal end of a second cannula into a side port of the cannulation coupler, inserting the distal end of the second cannula into the proximal end of the first cannula such that the distal end of the second cannula protrudes from the distal end of the first cannula; and securing the second cannula into the cannulation coupler may be accomplished in alternate order of operation and fashion according to surgical team preference and/or availability of individual components.

An alternate means of assembly of the coaxial coupler assembly may be to first engage the drainage cannula via the base port. The o-ring is pushed within the delivery cannula side port and the delivery cannula is then inserted via the side port of the cannulation coupling device and through the delivery cannula. When the desired position of the cannula is confirmed externally, the bushing is pushed into place and threaded on the main body of the coupling device to ensure a hemostatic seal. Finally, the appropriate tubing, indicated for use with the chosen ECMO pump, is engaged with the remaining side port of the device.

When all the appropriate components are present and the system is completely assembled, the patient will be cannulated for ECMO via the surgeon's preferred approach based on the given patient's indication for mechanical circulatory support (MCS). This is done using standard sterile technique. This system allows for the possibility of cannulating through the apex of the heart for left-sided cardiac support (VA-ECMO) or via the internal jugular vein for right-sided cardiac and/or ventilatory support (VV-ECMO). This system allows for independent repositioning of the drainage and delivery cannulae relative to one another. Once the proper location of the cannulae is verified, mechanical circulatory support is initiated. Deoxygenated blood will be transported via a 34-Fr drainage cannula, through the cannulation coupler and assembly, and to the chosen ECMO pump for oxygenation. At this point oxygenated blood will be pumped to the delivery cannula, contained within the drainage cannula, and will transport blood to the chosen great artery.

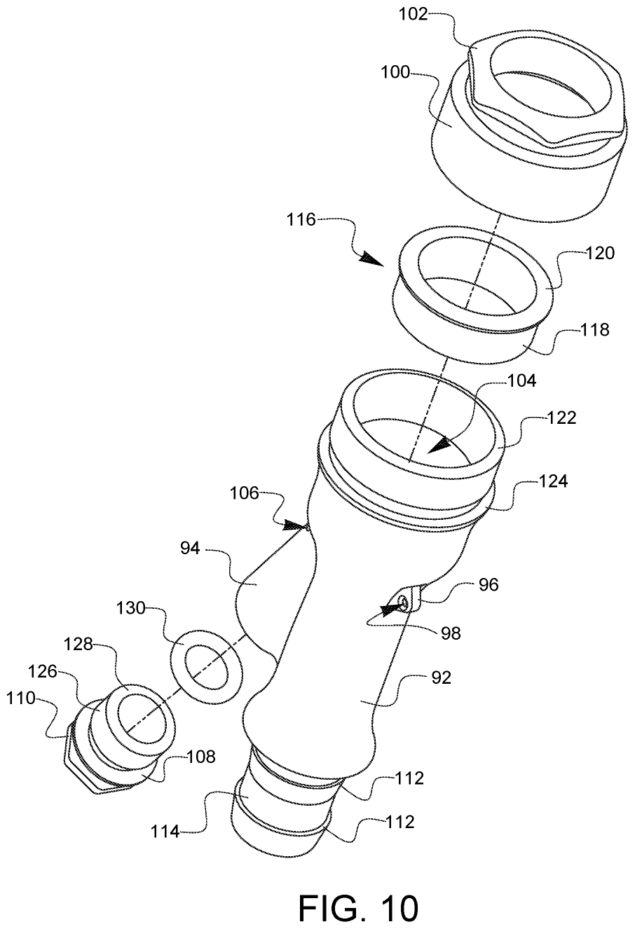

FIG. 10 is an exploded view illustrating assembly of the of the cannula coupler of FIG. 7. The cannulation coupler 90 defines a primary branch 92, having a primary opening 104. A bushing 116 having a flange 120 and an insert 118 to provide a depth limiter is inserted into the primary opening 104 of the primary branch 92. The base port cap 100 has inner threads, not shown in this view, that correspond to threads near the base port opening 104 of the cannulation coupler 90 and a head 102 having flat edges on the outer circumference of the base port cap 100. The flats on the head 102 provide a configuration for tightening of the base port cap 100 by mechanical tools, although the base port cap 100 may also be tightened by hand. The base port cap 100 is not fully tightened until a cannula or lumen is also inserted into the base port opening 104 once the bushing 116 and base port cap 100 are attached to the primary branch 92, as described in regard to FIGS. 9A-9C. Once fully tightened, the base port cap 100 and bushing 116 contribute to providing a leakproof and hermetically sealed connection for holding an outer dual lumen cannula providing a flow pathway through the connected outer first cannula. On an opposite end of the primary branch 92, the cannulation coupler 90 also defines an outflow branch 114 having several barbs 112. These barbs 112 are configured to temporarily yet reliably hold an outflow tube or lumen used in a VA-ECMO surgical assembly. On the side port 94 of the body of the cannulation coupler 90 are a set of inner threads, not shown in this view. The inner diameter of the inflow side port 94 is configured such that an o-ring 130 can be inserted into the side port 94 without the o-ring 130 falling into the opening of the inflow side port 94. There is a step or a ledge on the inner diameter of the side port 94. This feature is not shown here, but should be known to those skilled in the art. Once the o-ring 130 is in place inside the inflow opening of the side port 94, a side cap 108 having a head 110 and further defining a flange 128 and a side cap insert 126 is also placed in the side port 94. The flange 128 on the side cap 108 limits the depth of the insertion of the side cap 108 into the inflow opening of the side port 94. The side cap 108 also defines inner threads, which are not shown in this view, and several flats on the head 110, which are configured in a similar manner to that of the base port cap 100.

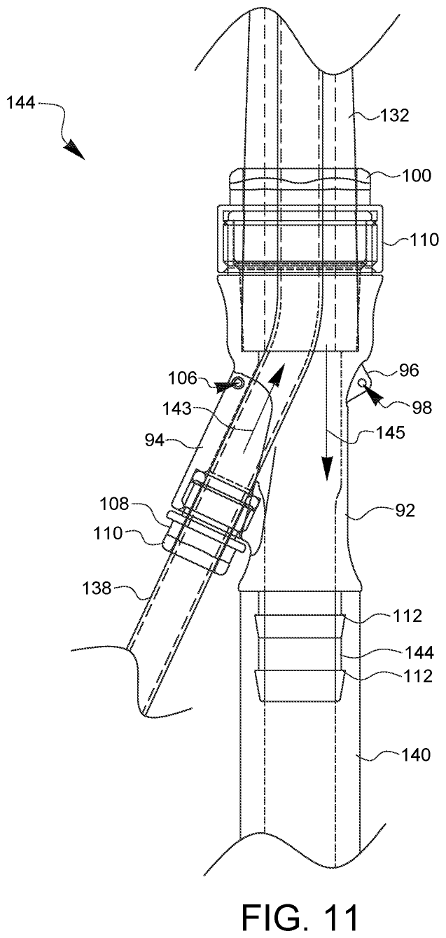

FIG. 11 is a schematic view of a surgical setting employing the cannula coupler assembly illustrated in FIGS. 9A-9C. FIG. 11 is a cross-sectional view of a cannulation assembly 144 utilizing the cannula coupler of FIG. 7. An outer first lumen 132 is shown inserted into the base port cap 100 and primary branch 92 end of the cannulation coupler 90 with the bushing 116 in place and the base port cap 100 tightened and sealed. An inner second lumen 138 is also shown inserted into the side port 94 of the cannulation coupler 90 with the o-ring 130 in place and the side cap 108 tightened and sealed. The inner second lumen 138 is further inserted into the outer first lumen 132 through the side port 94 and through the primary branch 92 and base port cap 100 in a dual lumen configuration. This could also be characterized as a coaxial dual lumen configuration or a cannula-in-cannula or lumen-in-lumen configuration. FIG. 11 also illustrates the inflow direction 143 of the cannulation assembly 144, which demonstrates the pathway and flow direction of the arterial blood into the aorta carried by the inner second lumen 138. Also illustrated is the pathway and outflow direction 145 of the venous or return flow carried by the outer first lumen 132 in an ECMO system.

The dual lumen coaxial cannulation assembly described herein results in an adjustable cannula intended towards ambulatory ECMO with a novel coupler. The cannulation system is intended for use in a minimally invasive transapical closure system, but may be applicable elsewhere. In many ECMO related procedures, the inflow or outflow pressures may or may not be monitored during the procedures. In some cases, only the flow rate of the inflow portion of the circuit is monitored during an ECMO procedure. A flow rate of 5 liters per minute is usually adequate for VA-ECMO. A target of 4.8-5.5 liters per minute is a common target and may be modified outside the stated boundaries relative to the treatment needs of a given patient, but 5 liters per minute is an adequate target value. V-V ECMO as introduced via an inner jugular vein may require flows as high as 6-7 liters per minute utilizing two separate 25 French cannulas. Pressure and flow are commonly the measurable criteria in perfusion technology related procedures.

In experimentation conducted using clinical ECMO oxygenation and pumping apparatus, control for flow was established using separate cannulas of the similar ranges of sizes as experimentally used. Near equivalent flow rates and pressures were observed when comparing a common ECMO setup using a 17 French inflow cannula and a 24 French outflow cannula with the coaxial dual lumen cannula assembly as described herein. The dimensions of the coaxial dual lumen were 17 French inflow or inner cannula inserted into a 34 French outer cannula. This phenomenon can be explained by the relationship between the cross-sectional diameters of the inner and outer cannula in the slidable coaxial dual lumen cannula assembly. The inflow cannula of the separate (non-coaxial) and coaxial cannula assemblies were both 17 French, or the same size. The outflow cannula of the non-coaxial cannula assembly was 24 French, while the outflow cannula of the coaxial cannula assembly was 34 French. The 34 French with the 17 French inner cannula inserted in the coaxial assembly results in restricted internal cross-sectional area and is comparable to a similar cross-sectional area in the 24 French outflow cannula in the non-coaxial cannula assembly. While these specific numbers are provided by way of illustrating the concept, they are not meant to be limited to only these dimensions of inflow and outflow cannulas, since the requirements of the system and patient condition may warrant the use or configuration of dual lumen coaxial cannula assemblies outside of the dimensions stated here by way of example.

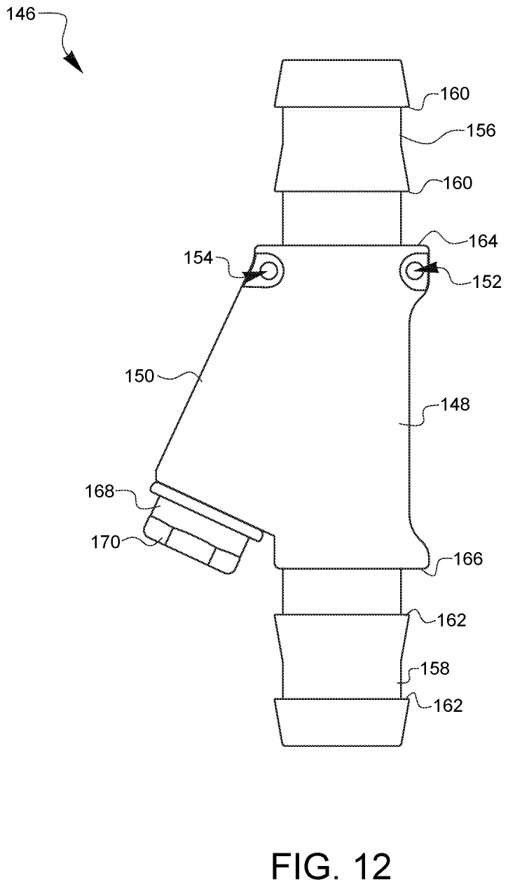

FIG. 12 is a perspective view of another embodiment of a cannulation coupler suitable for use in a single cannula dual lumen adjustable cannulation assembly for minimally invasive ambulatory ECMO. The cannulation coupler 146 has a primary branch 148 with a base port 156 in communication with a side inflow branch 150 and a drainage or external port, or outflow branch 158, each defined by the structure of the cannulation coupler 146. The primary branch 148 defines the base port 156 at one end which defines several concentric barbs 160 configured to securely yet releasably hold a lumen or cannula in place. The primary branch 148 also defines the outflow branch 158 at an opposite end which also defines several concentric barbs 162 also configured to securely yet releasably hold a lumen or cannula in place. The primary branch 148 also defines a base port limit 164 and an external port limit 166 at either end, configured to provide a sealing surface and consistent limitation for a lumen or cannula connected to either the base port 156 or the outflow branch 158. The primary branch 148 of the cannulation coupler 146 also defines several retaining features 152, 154 which are configured to anchor and secure the cannulation coupler 146 to a patient or to other apparatus used in an ambulatory ECMO procedure and treatment. The retaining features and the act of securing the cannulation coupler 146 and associated assembly to a patient enables and allows mobility of a patient while undergoing treatment under such procedures. One retaining feature 154 is adjacent to an outer junction between the side inflow branch 150 and the primary branch 148. On the side inflow branch 150, the cannulation coupler 146 also has a side inflow branch cap 168 which defines a head 170, the features of which have been previously described herein. The side inflow branch 150 further comprises helical threading, not shown in this view, on a portion of an inner circumference. As previously described herein, the side inflow branch cap 168 has an aperture or opening configured to pass through and hermetically seal there within a lumen or cannula as a part of the overall assembly. The cap 168 also has helical threads on an outer circumference that interlock with the inner threads on the side inflow branch 150. An axis defined by the side inflow branch 150 is disposed at an angle relative to an axis defined by the primary branch 148. While the angle illustrated in FIG. 12 is 25 degrees, alternate angles may be used in other embodiments. An axis defined by the outflow branch 158 is parallel to an axis defined by the primary branch. Alternate angles may be utilized in other embodiments of a cannulation coupler. The cannulation coupler 146 may be fabricated from a number of materials suitable for surgical use including surgical steel, plastic, or other suitable materials known in the art for transferring blood or similar fluids without interacting with the fluids unfavorably. It should be noted that the diameter of the side inflow branch 150 is smaller than the primary branch 148. Other embodiments of cannulation couplers may have a larger side inflow branch than the primary branch. Still other embodiments of cannulation couplers may have side inflow branch and primary branches having substantially similar diameters.

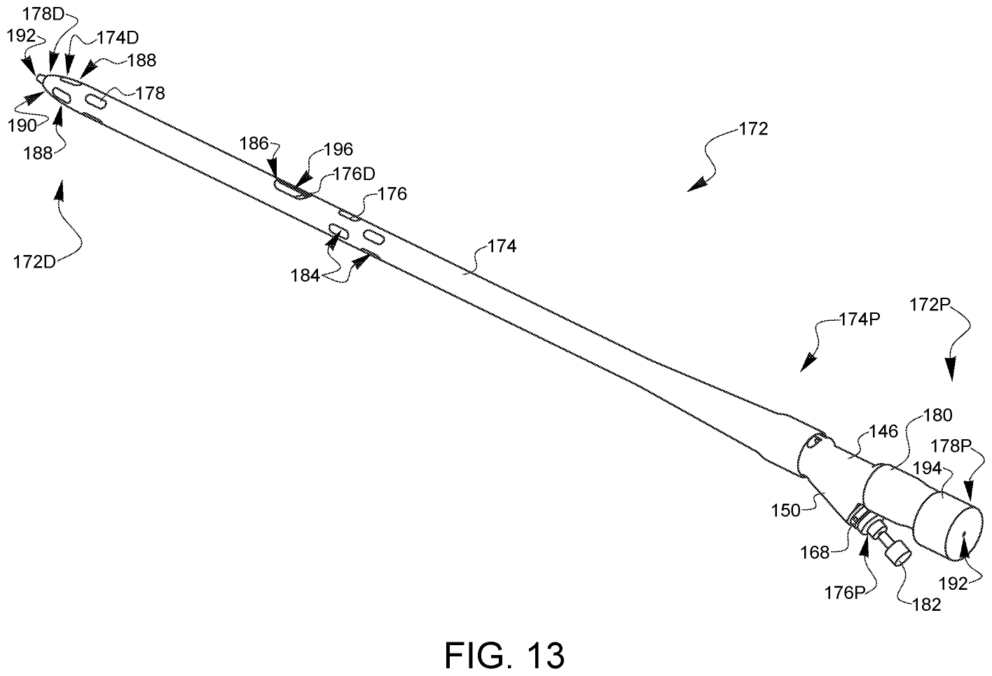

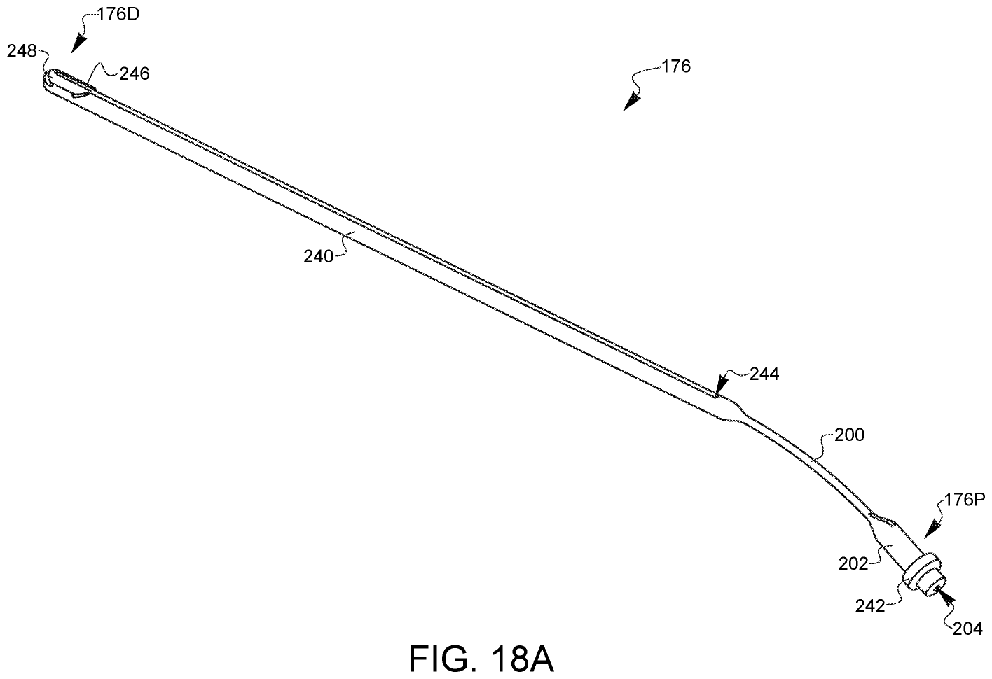

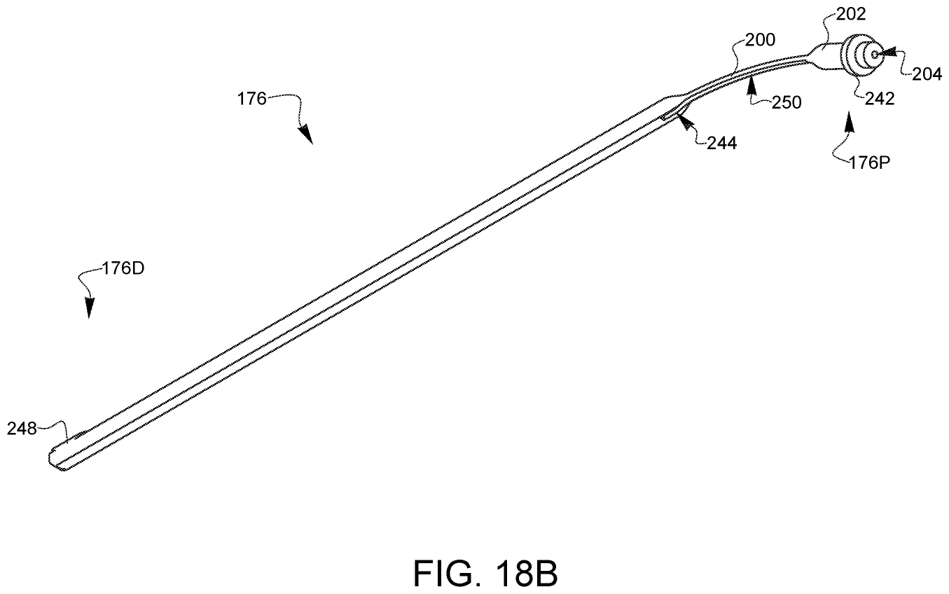

FIG. 13 is a top-left-front perspective view of an adjustable cannulation assembly. This embodiment of an adjustable cannulation assembly 172 includes a first IVC cannula 174 defining a first plurality of IVC perforations 188, a second plurality of upper SVC perforations 184, and a side port 186 in communication with an IVC cannula channel 190. The IVC cannula channel 190 of the first IVC cannula 174 continues from a distal end 174D of the first IVC cannula 174 to a proximal end 174P of the first IVC cannula 174. The side port 186 is configured such that it exits the IVC cannula channel 190 radially and is on a side of the first IVC cannula 174. The first IVC cannula 174 is coupled to the cannulation coupler 146 at the base port 156 of the cannulation coupler 146. On an opposite end of the cannulation coupler 146, an eccentric obturator cap 180 is coupled to the outflow branch 158 of the cannulation coupler 146. Inserted within the eccentric obturator cap 180 and continuing throughout the primary branch of the cannulation coupler 146, and further through the IVC cannula channel 190 of the first IVC cannula 174 is a first IVC obturator 178. The first IVC obturator 178 defines a knob 194 at a proximal end 178P of the first IVC obturator 178 and an obturator channel 192 from a proximal end 178P of the first IVC obturator 178 to a distal end 178D of the first IVC obturator 178. The distal end 178D of the first IVC obturator 178 is visible protruding from the distal end 174D of the first IVC cannula 174. Inserted within the side inflow branch cap 168 of the side inflow branch 150 of the cannulation coupler 146 is a pulmonary artery guidewire introducer 176. The pulmonary artery guidewire introducer 176 extends to the side port 186 of the first IVC cannula 174, where it terminates in a guidewire introducer exit 196 defined by a distal end 176D of the pulmonary artery guidewire introducer 176. A proximal end 176P of the pulmonary artery guidewire introducer 176 can be seen protruding from the side inflow branch cap 168 of the side inflow branch 150 on the cannulation coupler 146. A pulmonary artery guidewire introducer plug 182 is fitted into the proximal end 176P of the pulmonary artery guidewire introducer 176. The pulmonary artery guidewire introducer 176 will be discussed in further detail later in regard to FIGS. 18A and 18B.

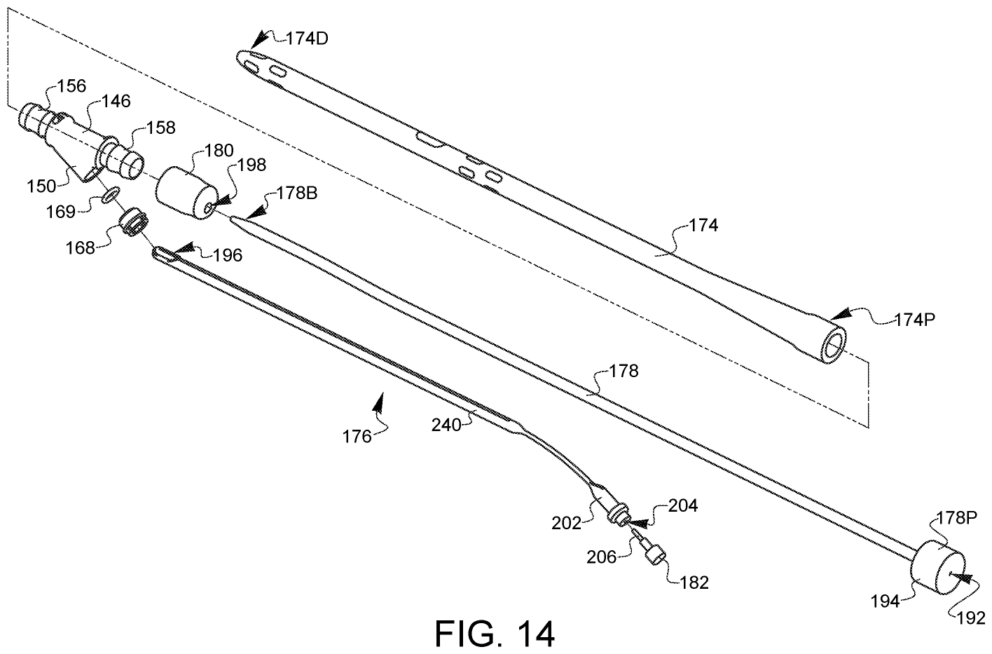

FIG. 14 is an exploded perspective view of the adjustable cannulation assembly of FIG. 13. The first IVC cannula 174 is coupled at the proximal end 174P to the base port 156 of the cannulation coupler 146, and held fixedly in place by the several barbs on the end of the base port 156. The o-ring 169 is placed into the side inflow branch 150 of the cannulation coupler 146, followed by the cap 168, which is screwed into the side inflow branch 150 over the o-ring 169. The eccentric obturator cap 180, of which an eccentric opening 198 is now visible, is attached to the external port 158 of the cannulation coupler 146, and held fixedly in place by the several barbs on the end of the external port 158. Next, the first IVC obturator 178 is inserted through the eccentric opening 198 of the eccentric obturator cap 180, and through the first IVC cannula 174 to the proximal end 174P of the first IVC cannula 174. It should be noted that the off center location of the eccentric opening 198 in the eccentric obturator cap 180 orients the first IVC obturator 178 towards one side of the first IVC cannula 174. Next, the pulmonary artery guidewire introducer 176, which further defines a body 240 portion, a neck 200 portion, and a fitting portion 202 is inserted by placing the distal end 176D of the pulmonary artery guidewire introducer 176 into the cap 168, through the side inflow branch 150 of the cannulation coupler 146, and finally into the first IVC cannula 174, terminating with the guidewire introducer exit 196 firmly positioned within the side port 186 of the first IVC cannula 174. Finally, the pulmonary artery guidewire introducer plug 182, which further defines an insert 206 portion, is releasably pressed into the entrance 204 of the pulmonary artery guidewire introducer 176.

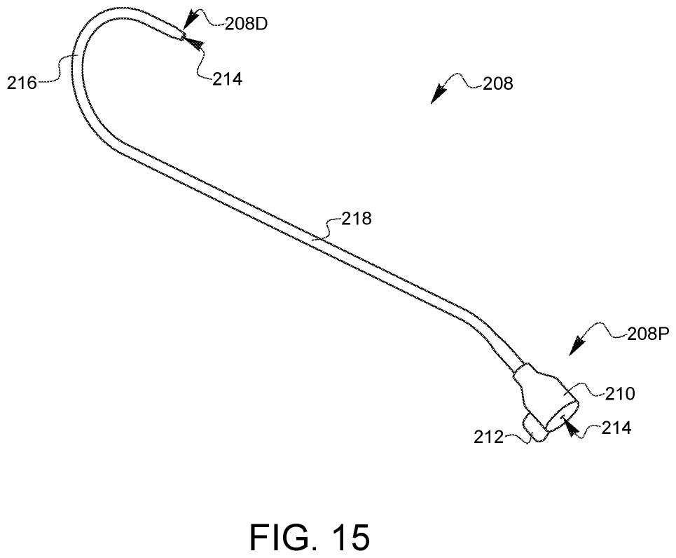

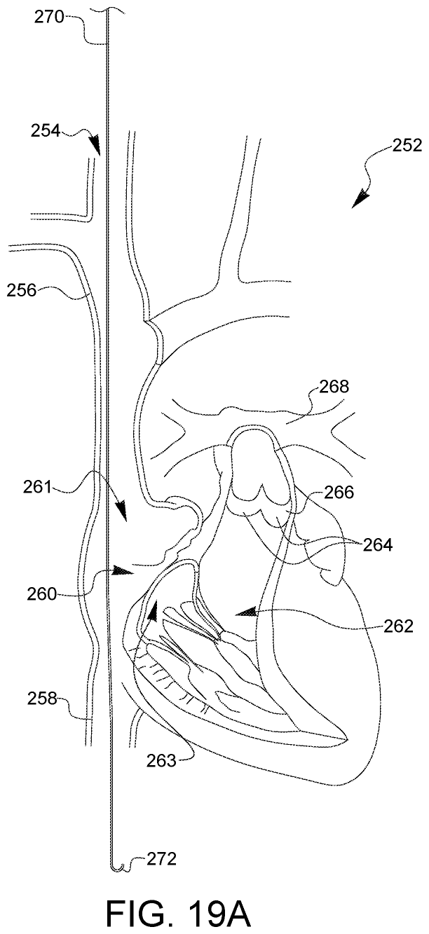

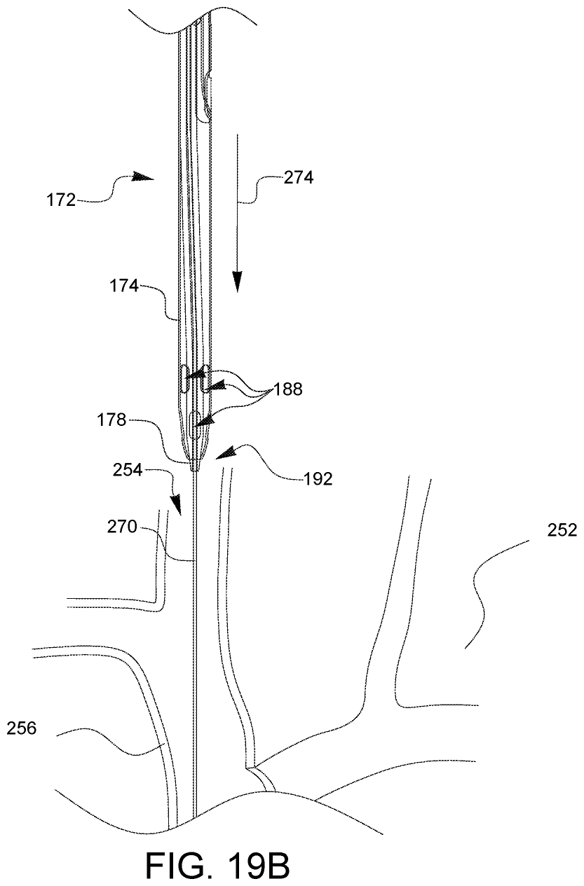

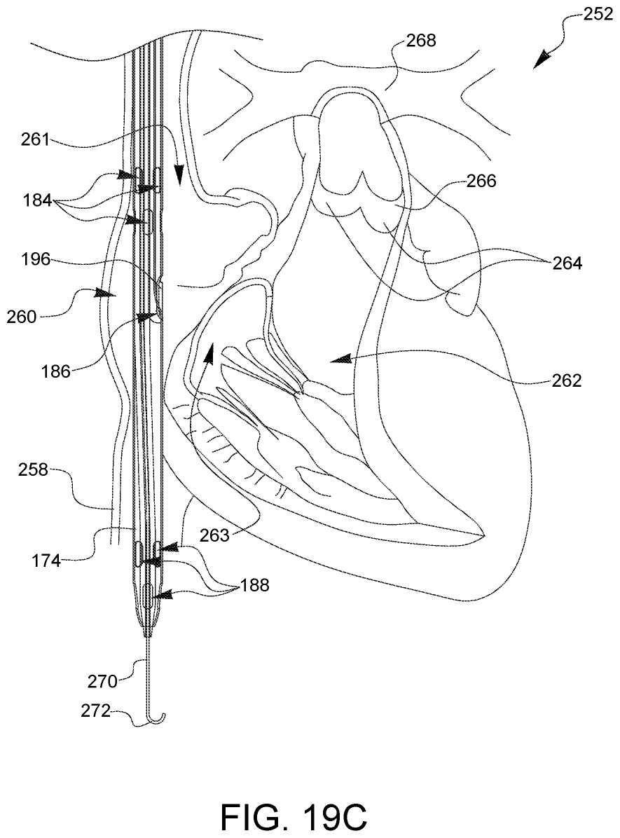

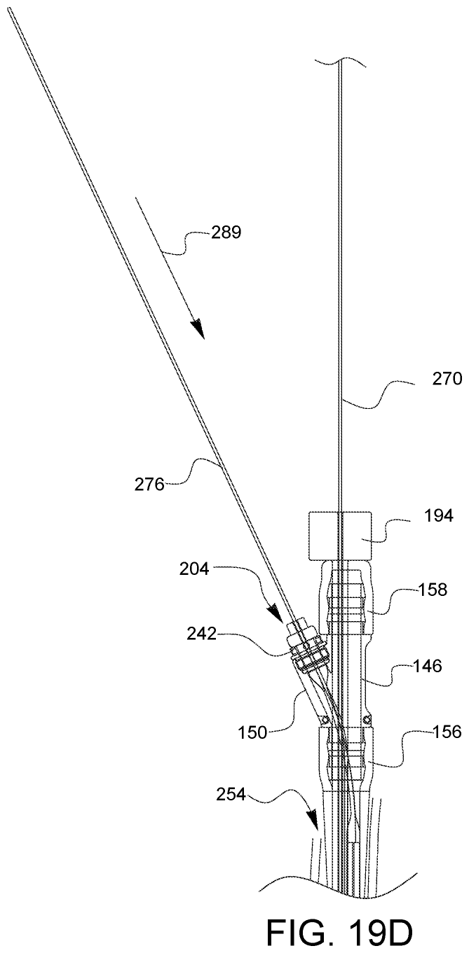

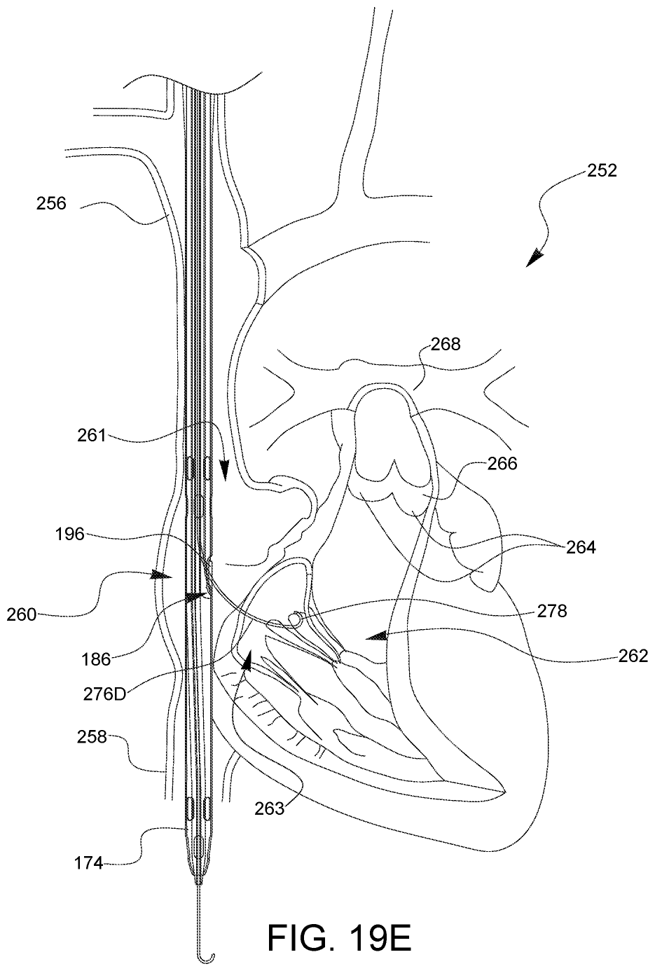

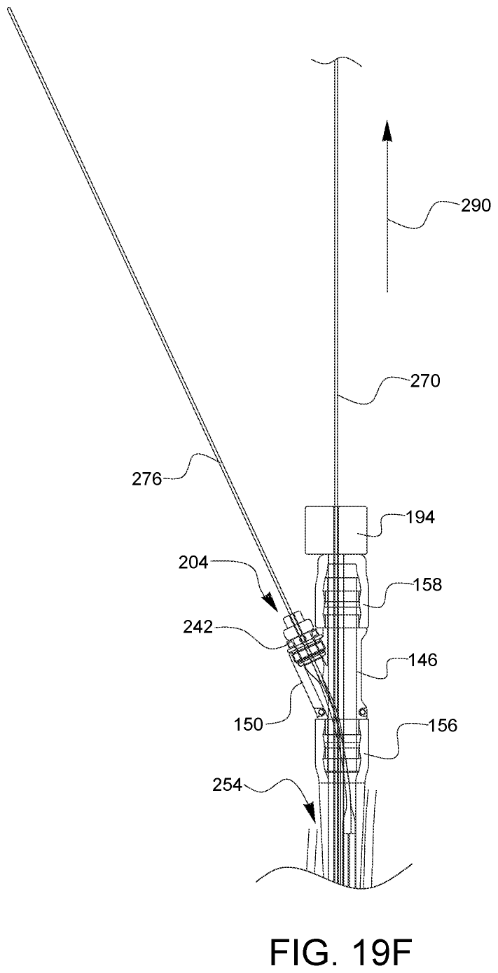

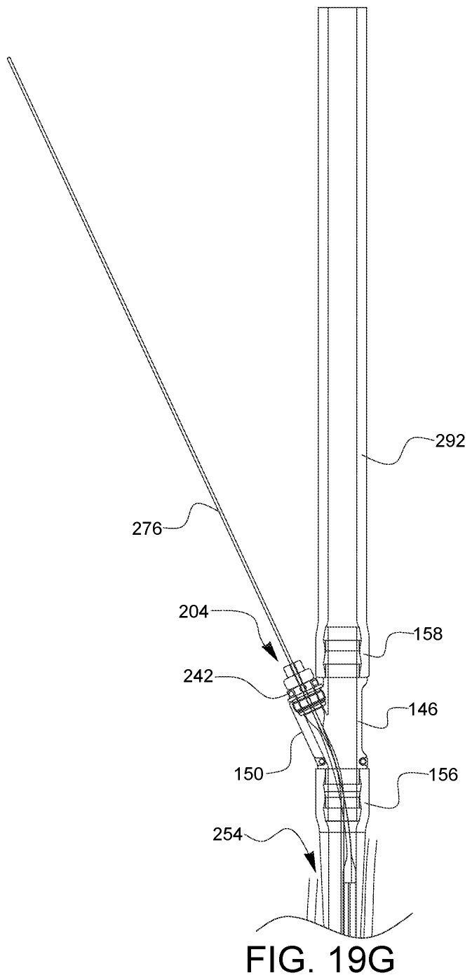

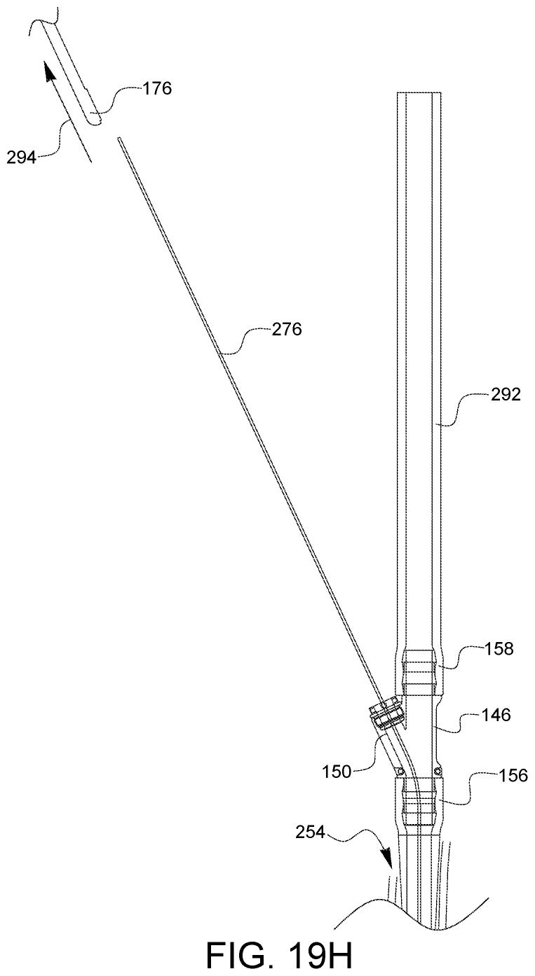

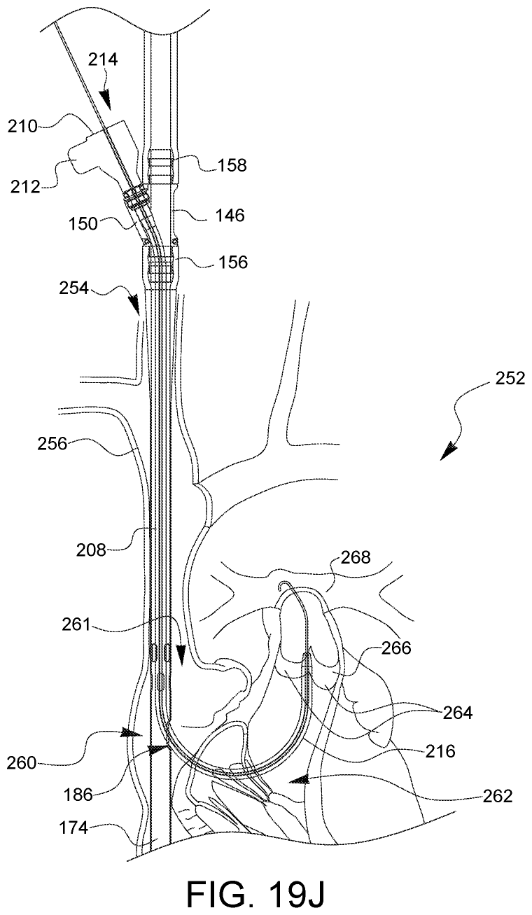

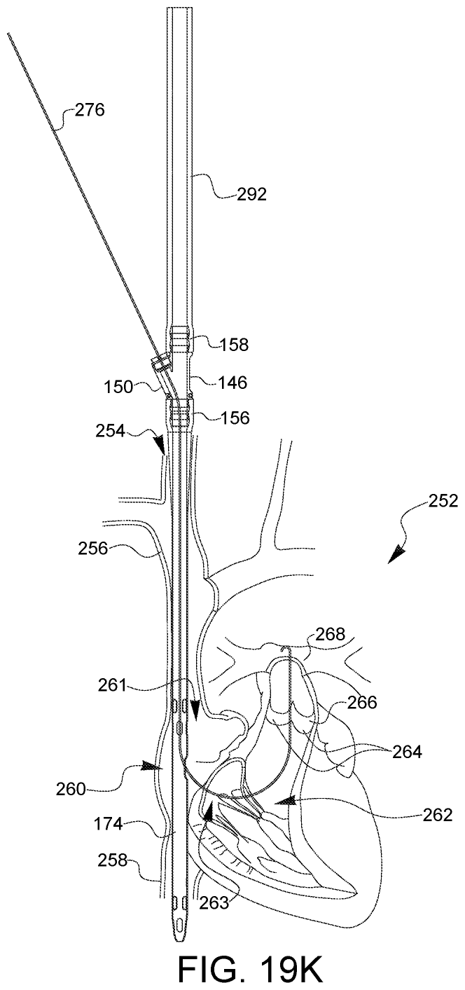

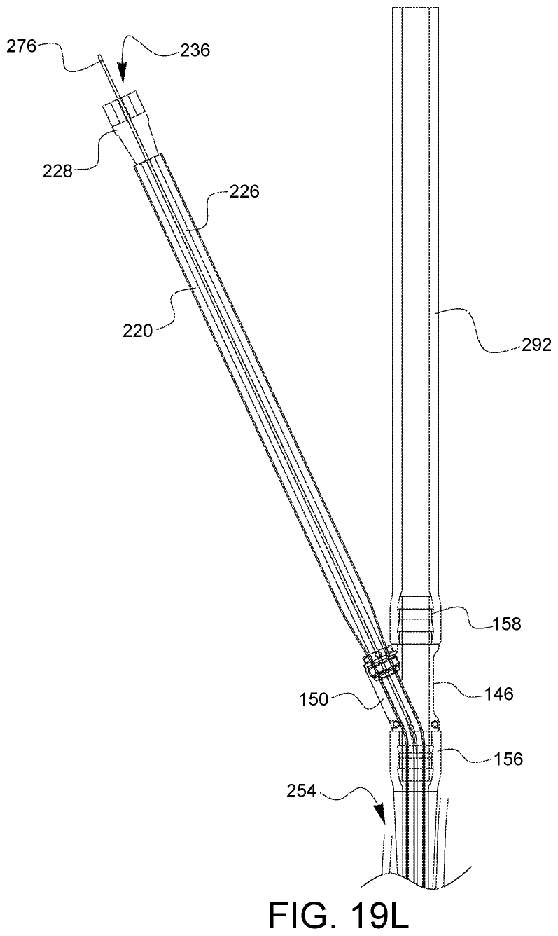

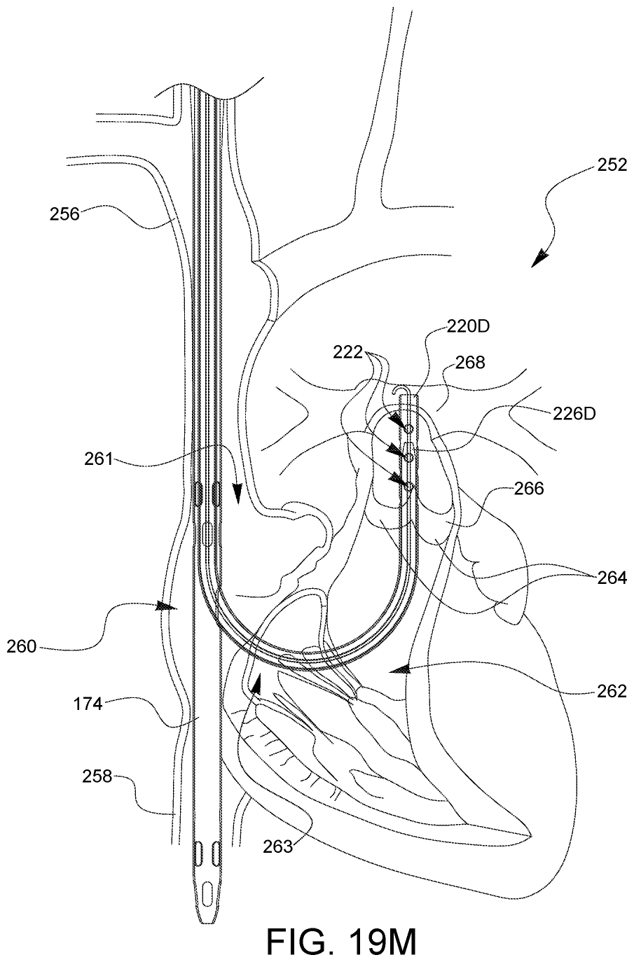

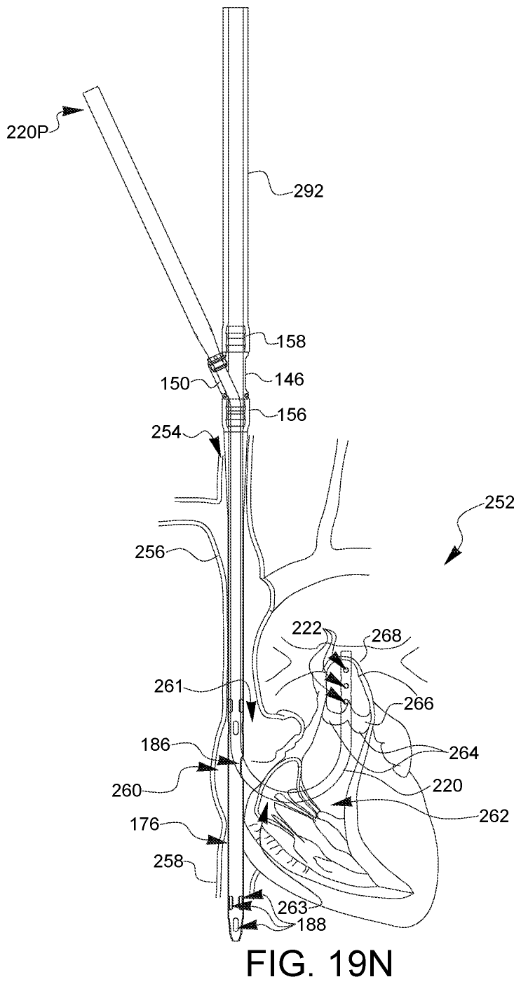

FIG. 15 is a top-left-front perspective view of a pulmonary artery guidewire director for use accompanying the adjustable cannulation assembly of FIG. 13. A pulmonary artery guidewire director 208 defines a knob 210 at a proximal end 208P. The knob 210 further defines a directional indicator fin 212, which is configured to enable turning or directionally orienting a pulmonary artery guidewire when the pulmonary artery guidewire director 208 is used within a minimally invasive procedure utilizing an adjustable cannulation assembly 172 as described herein. The pulmonary artery guidewire director 208 also defines a straight portion 218 and a curved portion 216 towards a distal end 208D of the pulmonary artery guidewire director 208. The pulmonary artery guidewire director 208 also has an inner channel 214, from the proximal end 208P to the distal end 208D of the pulmonary artery guidewire director 208 configured to guide and direct a guidewire therethrough. The pulmonary artery guidewire director 208 is comprised of a pre-curved flexible plastic material suitable for surgical use which can be introduced throughout a cannulation assembly such as the one described herein yet regain its shape upon exit of any constraint within a lumen or other delivery channel. While illustrated here as a preformed curved embodiment, alternate embodiments may be straight or otherwise shaped depending upon surgical preference, anatomical variations or other conditions, or configurations of accompanying guidewire structures or designs. The proposed use of the pulmonary artery guidewire director 208 will be described in further detail in regard to FIGS. 19A-19H and 19J-19N.

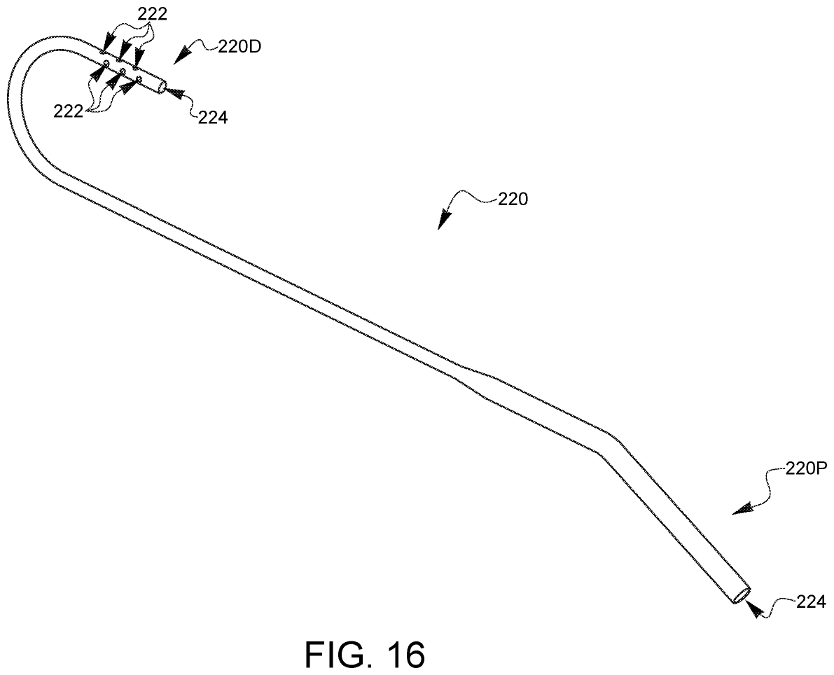

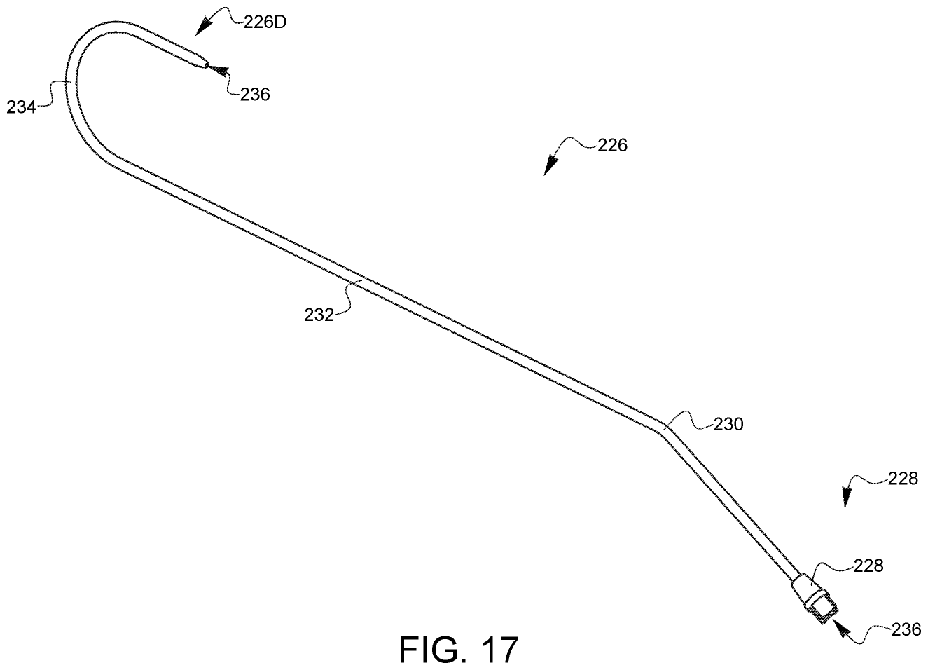

FIG. 16 is a top-left-front perspective view of an inner cannula for use accompanying the adjustable cannulation assembly of FIG. 13. An inner cannula 220 having a proximal end 220P and a distal end 220D also defines an inner channel 236 passing therethrough from the proximal end 220P to the distal end 220D, and several distal perforations 222 at the distal end 220D of the inner cannula 220. Typical commercially available cannulae used with an adjustable cannulation assembly as described herein will be a singular straight lumen, but the inner cannula 220 is shown in its desired state within the adjustable cannulation assembly 172 and as related to the procedures described for use within the adjustable cannulation assembly 172. The inner cannula 220 illustrated in FIG. 16 is sized as a 19 Fr cannula, but the specific size, additional design features, and configuration of the inner cannula used in minimally invasive surgical procedures described herein may be dependent upon the immediate surgical considerations as well as anatomical variations of a patient or available accompanying surgical equipment. The proposed use of the inner cannula 220 will be described in further detail in regard to FIGS. 19A-19H and 19J-19N.

FIG. 17 is a top-left-front perspective view of an inner cannula obturator for use accompanying the adjustable cannulation assembly of FIG. 13. An inner cannula obturator 226 having a proximal end 226P and a distal end 226D defines an inner channel 236 passing therethrough from the proximal end 226P to the distal end 226D of the inner cannula obturator 226. The inner channel 236 is configured to receive a guidewire such that the inner cannula obturator 226 may be utilized to help push and direct the inner cannula 220 through the adjustable cannulation assembly 172. The inner cannula obturator 226 also defines a //228 at the proximal end 226P. Alternate embodiments may have additional handle features such as a directional indicator. The inner cannula obturator 226 is illustrated as made from a pre-curved flexible plastic material suitable for surgical use that can be introduced throughout a cannulation assembly such as the one described herein, yet regain its shape upon exit of any constraint within a lumen or other delivery channel. While illustrated here as a preformed curved embodiment, alternate embodiments may be straight or otherwise shaped depending upon surgical preference, anatomical variations or other conditions, or configurations of accompanying guidewire structures or designs. The proposed use of the inner cannula obturator 226 for advancing an inner cannula through an adjustable cannulation assembly will be described in further detail in regard to FIGS. 19A-19H and 19J-19N.

FIGS. 18A and 18B are top-left-front and bottom-right-rear perspective views, respectively, of the pulmonary artery guidewire introducer of the adjustable cannulation assembly of FIG. 13. FIG. 18A illustrates the pulmonary artery guidewire introducer 176, which defines a fitting portion 202 and a cap 242 at a distal end 176D. The fitting portion 202 is sized and configured to fit within the cap of the cannulation coupler 146 in the adjustable cannulation assembly 172 of FIG. 13. Adjacent to the proximal end 176P is a neck 200 coupled to the fitting portion 202 and a body 240 coupled to the neck 200. Along the body 240 of the pulmonary artery guidewire introducer 176 is a second side channel 246 which begins at an aperture 244 near the junction between the neck 200 and body 240 and terminates at a side port interlock feature 248 at a distal end 176D of the pulmonary artery guidewire introducer 176. The side port interlock feature 248 is sized and configured to interface in a complementary fashion with the side port 186 of the first IVC cannula 174 within the adjustable cannulation assembly 172. This feature ensures that the pulmonary artery guidewire introducer 176 is correctly positioned within the first IVC cannula 174 and that a guidewire inserted into and through the pulmonary artery guidewire introducer 176 will be reliably directed outward from the side port interlock feature 248 portion of the pulmonary artery guidewire introducer 176 and out of the side port 186 of the adjustable cannulation assembly 172. FIG. 18B is a bottom-right-rear perspective view of the pulmonary artery guidewire introducer 176 and illustrates a first side channel 250 located within the neck 200 portion of the pulmonary artery guidewire introducer 176. The pulmonary artery guidewire introducer 176 is configured to receive and direct a guidewire from the entrance 204 at the proximal end 176P into the first side channel 250, through the aperture 244 to the opposite side of the pulmonary artery guidewire introducer 176, through the second side channel 246 as shown in FIG. 18A, and out of the side port interlock feature 248 at the distal end 176D of the pulmonary artery guidewire introducer 176. The pulmonary artery guidewire introducer 176 is made from a flexible, surgical grade plastic or other material and may also have an enclosing tube-like structure, collar, or other similar retention feature coupled around the neck 200 to further entrain and guide a guidewire inserted throughout the pulmonary artery guidewire introducer 176 as described. The intended use of the pulmonary artery guidewire introducer 176 within the adjustable cannulation assembly 172 will be described in further detail in regard to FIGS. 19A-19H and 19J-19N.

FIGS. 19A-19H and 19J-19N are schematic illustrations of a surgical method for use of the adjustable cannulation assembly of FIG. 13 with the additional components of FIG. 15, FIG. 16, and FIG. 17. It should be noted that FIG. 19I was not used so as not to be confused with the number 191. While previously described embodiments of adjustable cannulation assemblies, for example, as described in regard to FIGS. 1-10 may have been utilized in a minimally invasive surgical procedure involving a transapical entry position, alternate minimally invasive approaches that still preserve patient mobility and ambulatory accommodation may be employed. For example, utilization of an adjustable cannulation assembly having five ports such as the embodiment illustrated in FIG. 13 via access or entry via the right internal jugular vein (IJ) may be used based on the immediate needs of a particular patient, surgical team preference, accessory equipment availability, or combinations thereof. Preparation of a patient for use of an adjustable cannulation assembly via the right internal jugular vein (IJ) includes establishing a patient in a prone Trendelenburg position with legs elevated and head down. The skin centered over the right IJ is prepared, a standard skin incision and superficial dissection is performed, using direct pressure tamponade as needed, and ultrasound guidance is used to perform a needle puncture of the right IJ vein. Needle tip location may be confirmed using blood aspiration. While these steps are not explicitly illustrated herein, they should be well-known to one skilled in the art. As illustrated in FIG. 19A, once a patient is prepared for a right inner jugular access ECMO cannulation procedure, an IVC guidewire 270 is placed into the inner jugular entry 254 through a superior vena cava 256 portion of a patient's heart 252, and down through to an inferior vena cava 258, using ultrasound guidance, and optionally, the use of a snare from the groin if necessary. Other features of the heart are illustrated herein, including their approximate relative locations, including a right atrium 260, right ventricle 262, tricuspid valve 263, pulmonary valve leaflets 264, pulmonary valve 266, and pulmonary artery 268.

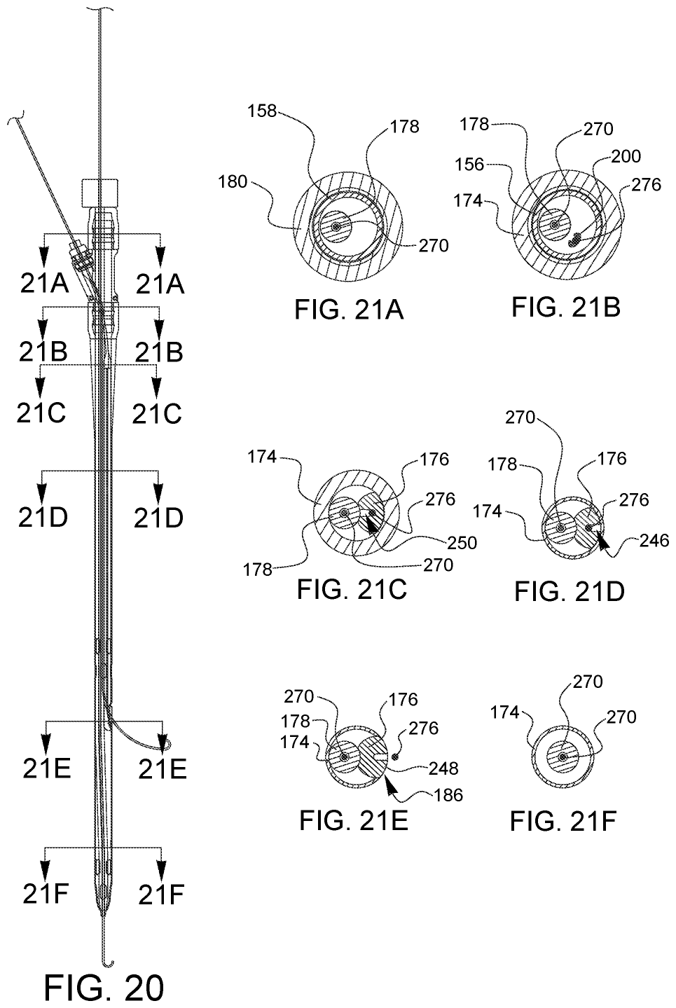

To proceed with inferior vena cava cannulation, the wound site external to the patient is then serially dilated to accommodate a 30-Fr outer diameter tube. The adjustable cannulation assembly 172 is prepared for use and flushed with saline. As illustrated in FIG. 19B, the IVC guidewire 270 is passed into the conical tip of the first IVC obturator 178 at the distal end 172D of the adjustable cannulation assembly 172. The adjustable cannulation assembly 172 is then advanced in direction 274 towards the inferior vena cava 258 until the lower IVC perforations 188 in the IVC cannula 174 are seen by ultrasound in the IVC. This intended position is shown in FIG. 19C. Right ventricle cannulation is then accomplished by first removing the pulmonary artery guidewire introducer plug 182 from the pulmonary artery guidewire introducer 176 located in the side inflow branch 150 of the cannulation coupler 146. A pulmonary artery guidewire 276 is then introduced into the entrance 204 of the pulmonary artery guidewire introducer 176 in direction 289, as illustrated in FIG. 19D. FIG. 19E shows the intended location of a j-tip 278 at a distal end of the pulmonary artery guidewire 276 as directed by the first side channel and second side channel of the pulmonary artery guidewire introducer 176, as previously described in regard to FIGS. 18A and 18B. The j-tip 278 of the pulmonary artery guidewire 276 exits through the side port 186 and into the right atrium 260. Further details describing the pulmonary artery guidewire 276 pathway through the pulmonary artery guidewire introducer 176 and through the adjustable cannulation assembly 172 will be further described in regard to FIG. 20 and FIGS. 21A-21F. At this point the rotational position should be established and confirmed, such that the side inflow branch 150 of the cannulation coupler 146 is directed away from the patient's chin and neck. The pulmonary artery guidewire 276 may be advanced and retracted to aim the j-tip 278 towards the tricuspid valve 263. The j-tip 278 is then advanced through the tricuspid valve 263 and into the right ventricle 262. Once the pulmonary artery guidewire 276 is in position in the right ventricle 262 as illustrated in FIG. 19E, the IVC obturator 178 and the IVC guidewire 270 are removed from the external port 158 of the cannulation coupler 146 by retracting in direction 290, as illustrated in FIG. 19F. Next, as shown in FIG. 19G, an ECMO drainage tube 292 is attached to the external port 158 of the cannulation coupler 146 and secured as needed. FIG. 19H illustrates the removal of the pulmonary artery guidewire introducer 176 via direction 294 and removing the pulmonary artery guidewire 276 from the internal channels of the pulmonary artery guidewire introducer 176, leaving the j-tip 278 of the pulmonary artery guidewire 276 in the right ventricle 262.

Cannulation of the pulmonary artery is then accomplished by advancing the flexible preformed pulmonary artery guidewire director 208 over the pulmonary artery guidewire 276 and into the side inflow branch 150 of the cannulation coupler 146 until the radiopaque distal end exits the side port 186 and enters the right ventricle 262. Alternatively, a steerable or pre-angled guidewire may be used in place of the pulmonary artery guidewire director 208. The pulmonary artery guidewire director 208 is manipulated, along with its indwelling pulmonary artery guidewire 276, by use of the directional indicator fin 212 to pass the pulmonary artery guidewire 276 distal to the pulmonic valve 262. Alternatively, if indicated, this pulmonary artery guidewire 276 may be replaced with a larger caliber, more rigid, or otherwise configured guidewire. The final placement of this pulmonary artery guidewire director 208 and location of the pulmonary artery guidewire 276 are illustrated in FIG. 19J. Once the j-tip 278 of the pulmonary artery guidewire 276 is in either the left or right branch of the pulmonary artery 268 the pulmonary artery guidewire director 208 is removed. The pulmonary artery guidewire director 208 is shown removed and the j-tip 278 of the pulmonary artery guidewire 276 is located in the pulmonary artery 268 in FIG. 19K. A flushed inner delivery cannula 220 with the in-place inner cannula obturator 226 over the pulmonary artery guidewire 276 is advanced through the side inflow branch 150 in the cannulation coupler 146 as shown in FIG. 19L, and through the adjustable cannulation assembly 172 until exiting the side port 186 as shown in FIG. 19M. While a 19-Fr inner cannula is shown in use, other sizes or configurations may be used as dictated by surgical preference or patient anatomy. The inner cannula 220 is advanced over the pulmonary artery guidewire 276 until all of the distal perforations 222 are distal to the coapted pulmonary valve leaflets in the pulmonary valve 266. Achieving this may depend on elements described herein having varying dimensions, additional tools, or complimentary techniques not described herein, but known to those skilled in the art, and may depend on patient anatomy or other considerations. The inner cannula obturator 226 and the pulmonary artery guidewire 276 are then removed from the adjustable cannulation assembly 172 via the side inflow branch 150 of the cannulation coupler 146 as illustrated in FIG. 19N. Once in this configuration, an inflow ECMO tube is attached or coupled to the inner cannula 220 and secured as needed. The adjustable cannulation assembly 172 is now vented and air-free flow is established, the optimal locations of the lower IVC perforations 188 and upper SVC perforations 184 in the IVC cannula 174 are reconfirmed, and the cannulation coupler 146 is secured to the skin near the initial puncture site to establish relative location of the adjustable cannulation assembly 172. Supra-valvular positioning of all distal perforations 222 in the inner delivery cannula 220 and appropriate flow rates and pressures are reconfirmed. The ECMO circuit has now been established. A wrench or other suitable tool is then used to tighten and lock the nut cap on the cannulation coupler 146. Finally, the procedure concludes with confirmation of cannula position flows and pressures, taping exposed assembly connections to further cover and secure components outside of the patient's body, and dressing the wound site.

FIG. 20 is a side view of the adjustable cannulation assembly of FIG. 13 illustrating several locations along a path followed by a directed pulmonary artery guidewire through the adjustable cannulation assembly. FIGS. 21A-21F are a series of several cross-sectional views of the adjustable cannulation assembly indicated in FIG. 20. The cross-sections follow the path of the pulmonary artery guidewire from the side inflow branch 150 of the cannulation coupler 146 down through the IVC cannula 174 and out of the side port 186 of the IVC cannula 174. While procedurally, the elements shown in FIGS. 21A-21F may not be inserted within the assembly at the same time, these cross-sections are intended to be descriptive of the path of the pulmonary artery guidewire 276 through the adjustable cannulation assembly 172 as directed primarily by the pulmonary artery guidewire introducer 176. The cross-section illustrated in FIG. 21A shows the respective locations of the IVC guidewire 270 within the IVC obturator 178. The eccentric opening in the eccentric obturator cap 180 is located such that the first IVC obturator 178 is close to the wall of the external port 158 of the cannulation coupler 146 when inserted into the adjustable cannulation assembly 172. Moving towards the cross-section illustrated in FIG. 21B, the neck 200 of the pulmonary artery guidewire introducer 176 and the placement of the pulmonary artery guidewire 276 positioned within is shown in its location relative to the first IVC obturator 178 within the base port 156 of the cannulation coupler 146. The cross-section illustrated in FIG. 21C shows the location of the IVC obturator 178 and the pulmonary artery guidewire introducer 176 within the IVC cannula 174, along with the respective locations of the IVC guidewire 270 and pulmonary artery guidewire 276. The complimentary shape of the body 240 portion of the pulmonary artery guidewire introducer 176 compared to the outer circumference of the IVC obturator 178 ensures proper orientation and direction of the pulmonary artery guidewire 276 through the adjustable cannulation assembly 172. The neck 200 configuration, combined with the complimentary shape and contour of the body 240 portion of the pulmonary artery guidewire introducer 176 allow the pulmonary artery guidewire introducer 176 to be inserted into the adjustable cannulation assembly 172 in a manner that enables the pulmonary artery guidewire 276 to be inserted into the side inflow branch 150 of the cannulation coupler 146 and pass around the outer circumference of the IVC obturator 178 in order to exit from the side port 186 of the IVC cannula 174 of the adjustable cannulation assembly 172. The cross-section illustrated in FIG. 21D illustrates a position where the pulmonary artery guidewire 276 has been passed through the aperture 244 within the pulmonary artery guidewire introducer 176, as previously described in regard to FIGS. 18A-18B, and has moved from the first side channel 250 in the neck 200 over through the aperture 244 to the second side channel 246 within the pulmonary artery guidewire introducer 176. The cross-section illustrated in FIG. 21E shows a position further down the adjustable cannulation assembly 172 where the side port interlock feature 248 of the pulmonary artery guidewire introducer 176 meets the side port 186 of the IVC cannula 174, and the pulmonary artery guidewire 276 exits the side port interlock feature 248 and the side port 186 into the right atrium 260 as first described in regard to FIG. 19E. The cross-section illustrated in FIG. 21F shows a position below the side port 186 on the IVC cannula 174, where the IVC obturator 178 is no longer positionally constrained by either the pulmonary artery guidewire introducer 176 or the eccentric opening in the eccentric obturator cap 180.

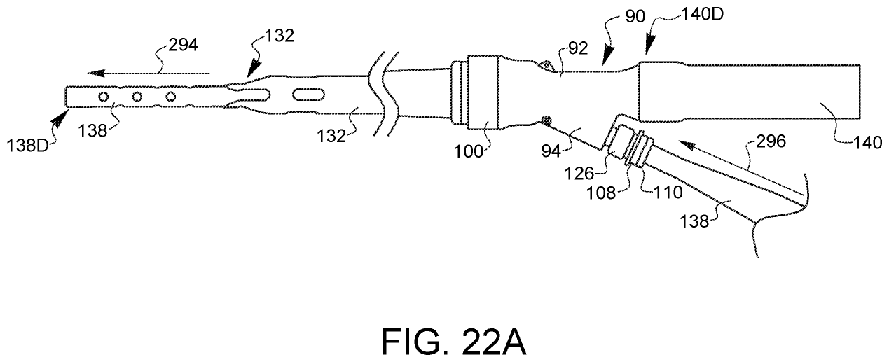

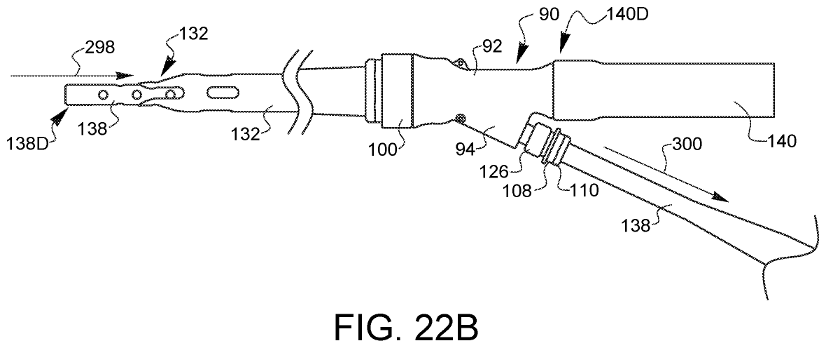

FIGS. 22A and 22B are side views of the embodiment of an adjustable cannulation assembly discussed previously in FIG. 9A-9C, illustrating adjustment of the second lumen 138 relative to the first lumen 132. One benefit of the system and approach described herein is that the relative position of the lumen 138, 132 can be adjusted for each unique patient anatomy. In other words, the second lumen 138 does not need to stick out a fixed distance from the first lumen 132. As shown in FIG. 22A, the second lumen 138 may be extended 294 relative to the first lumen 132. Similarly, as shown in FIG. 22B, the second lumen 138 may be retracted relative to the first lumen 132.

Various advantages of an adjustable cannulation assembly and methods thereof have been discussed above. Embodiments discussed herein have been described by way of example in this specification. It will be apparent to those skilled in the art that the foregoing detailed disclosure is intended to be presented by way of example only, and is not limiting. As just one example, although the end effectors in the discussed examples were often focused on the use of a scope, such systems could be used to position other types of surgical equipment. Various alterations, improvements, and modifications will occur and are intended to those skilled in the art, though not expressly stated herein. These alterations, improvements, and modifications are intended to be suggested hereby, and are within the spirit and the scope of the claimed invention. The drawings included herein are not necessarily drawn to scale. Additionally, the recited order of processing elements or sequences, or the use of numbers, letters, or other designations therefore, is not intended to limit the claims to any order, except as may be specified in the claims. Accordingly, the invention is limited only by the following claims and equivalents thereto.

What is claimed is:

1. A cannulation coupler, comprising:
a primary branch comprising a retaining feature;
a side inflow branch in communication with the primary branch; and
an outflow branch in communication with the primary branch and in communication with the side inflow branch.

2. The cannulation coupler of claim 1, wherein the retaining feature is disposed on an outer circumference of the primary branch.

3. The cannulation coupler of claim 1, further comprising a retaining feature adjacent to an outer junction between the side inflow branch and the primary branch.

4. The cannulation coupler of claim 1, wherein the side inflow branch is partially cylindrical.

5. The cannulation coupler of claim 4, wherein the side inflow branch extends in a proximal direction away from the primary branch.

6. The cannulation coupler of claim 1, wherein the side inflow branch further comprises helical threading on a portion of an inner circumference.

7. The cannulation coupler of claim 1, wherein the side inflow branch further comprises a seat configured to hold a sealing element.

8. The cannulation coupler of claim 1, further comprising a cap having helical threads on an outer circumference.

9. The cannulation coupler of claim 8, wherein the cap further comprises an aperture.

10. The cannulation coupler of claim 1, wherein an axis defined by the side inflow branch is disposed at an angle relative to an axis defined by the primary branch.

11. The cannulation coupler of claim 8, wherein the angle of an axis defined by the side inflow branch relative to an axis defined by the primary branch is 25 degrees.

12. The cannulation coupler of claim 1, wherein an axis defined by the outflow branch is parallel to an axis defined by the primary branch.

13. The cannulation coupler of claim 1, wherein a diameter of the side inflow branch is smaller than a diameter of the outflow branch.

14. The cannulation coupler of claim 1, wherein a diameter of the side inflow branch is smaller than a diameter of the primary branch.

15. An adjustable cannulation assembly, comprising:
a cannulation coupler, comprising:
a primary branch comprising a retaining feature;
a side inflow branch in communication with the primary branch; and
an outflow branch in communication with the primary branch and in communication with the side inflow branch;
an outer cannula coupled to the primary branch; and
an inner cannula coupled to the side inflow branch, wherein:
the inner cannula also passes through the primary branch; and
the inner cannula is coaxially slidable within the outer cannula.

16. An adjustable cannulation assembly, comprising:
a cannulation coupler, comprising a primary branch, a side inflow branch in communication with the primary branch, and an outflow branch in communication with the primary branch and in communication with the side inflow branch;
a first cannula coupled to the primary branch;
an obturator cap coupled to the outflow branch; and
an obturator inserted into the obturator cap and through the outflow branch, the primary branch, and the first cannula.

17. The adjustable cannulation assembly of claim 16, wherein the cannulation coupler further comprises a retaining feature.

18. The adjustable cannulation assembly of claim 16, wherein the cannulation coupler further comprises a cap defining an aperture.

19. The adjustable cannulation assembly of claim 16, wherein the first cannula further comprises a first plurality of perforations at a distal end.

20. The adjustable cannulation assembly of claim 19, wherein the first cannula further comprises a second plurality of perforations.

21. The adjustable cannulation assembly of claim 20, wherein the first cannula further comprises a side port opening radially from a longitudinal axis of the first cannula.

22. The adjustable cannulation assembly of claim 21, wherein the side port is located between the first plurality of perforations and the second plurality of perforations.

23. The adjustable cannulation assembly of claim 16, wherein the obturator cap has an eccentric opening

24. The adjustable cannulation assembly of claim 16, wherein the obturator further comprises an internal channel.

25. The adjustable cannulation assembly of claim 16, further comprising a guidewire introducer inserted into the side inflow branch of the cannulation coupler.

26. The adjustable cannulation assembly of claim 25, wherein the guidewire introducer further comprises a first channel on a first side of the guidewire introducer and a second channel on a second side of the guidewire introducer.

27. The adjustable cannulation assembly of claim 26, wherein the guidewire introducer further comprises an aperture between the first channel and the second channel.

28. The adjustable cannulation assembly of claim 25, wherein the guidewire introducer further comprises an interlocking feature at a distal end.

29. The adjustable cannulation assembly of claim 16, further comprising a guidewire director inserted into the side inflow branch of the cannulation coupler.

30. The adjustable cannulation assembly of claim 29, wherein the guidewire director further comprises a directional indicator fin.

31. The adjustable cannulation assembly of claim 29, wherein the guidewire director further comprises an internal channel.

32. The adjustable cannulation assembly of claim 29, wherein the guidewire director further comprises a preformed curve portion.

33. The adjustable cannulation assembly of claim 16, further comprising a second inner cannula inserted into the side inflow branch of the cannulation coupler.

34. The adjustable cannulation assembly of claim 16, further comprising an inner cannula obturator inserted into the side inflow branch of the cannulation coupler.

35. A method of assembling an adjustable cannulation assembly, comprising:
removing a section from a proximal end of a first cannula;
removing a section from a distal tip of the first cannula;
affixing the proximal end of the first cannula onto a main port of a cannulation coupler;
inserting a distal end of a second cannula into a side port of the cannulation coupler;
inserting the distal end of the second cannula into the proximal end of the first cannula such that the distal end of the second cannula protrudes from the distal end of the first cannula; and
securing the second cannula into the cannulation coupler.

36. A method of cannulation with an adjustable cannulation assembly, comprising:
placing a first guidewire into an inner jugular entry through to an inferior vena cava;
passing the adjustable cannulation assembly over the first guidewire into the inferior vena cava;
passing a second guidewire into a side port of the adjustable cannulation assembly, out through a side port of the adjustable cannulation assembly and into a right atrium;
advancing the second guidewire through a tricuspid valve and into a right ventricle;
removing the first guidewire from the adjustable cannulation assembly;
attaching an external drainage tube to an external port on the adjustable cannulation assembly;
advancing the second guidewire into a pulmonary artery;
advancing an inner delivery cannula over the second guidewire and into the pulmonary artery until several perforations in the inner delivery cannula are distal to a coapted pulmonary valve; and
attaching an inflow tube to a proximal end of the inner delivery cannula.
